(19) United States
(12) Patent Application Publication
(10) Pub. No.: US 2010/0022549 A1
(43) Pub. Date: Jan. 28, 2010

(54) ALPHA-HELIX MIMETIC WITH FUNCTIONALIZED PYRIDAZINE

(75) Inventors: Julius Rebek, JR., La Jolla, CA (US); Lionel Moisan, Gif-Sur-Yvette Cedex (FR); Alexandre Carella, Grenoble Cedex (FR); Enrique Mann, Madrid (ES)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Scripps Research Institute, La Jolla, CA (US)

(21) Appl. No.: 12/508,419

(22) Filed: Jul. 23, 2009

Related U.S. Application Data
(60) Provisional application No. 61/137,050, filed on Jul. 23, 2008.

(51) Int. Cl. A61K 31/501; C07D 403/14; C07K 1/00

(52) U.S. Cl. 514/252.02; 544/238; 530/402

(57) ABSTRACT

The synthesis of new α-helix scaffolds mimicking i, i+3 or i+4, i+7 residues, was accomplished. The common pyridazine heterocycle originates from the easily available dimethyl pyridazine-3,6-dicarboxylate building block. These scaffolds may be thought of as synthetic counterparts of amphiphilic α-helices having a hydrophilic face along one side and a hydrophobic face along the other side of the helix.

### CROSS-REFERENCES TO RELATED APPLICATIONS

This application claims the benefit of U.S. Provisional Application No. 61/137,050, filed Jul. 23, 2008, which is incorporated herein by reference in its entirety and for all purposes.

### STATEMENT AS TO RIGHTS TO INVENTIONS MADE UNDER FEDERALLY SPONSORED RESEARCH OR DEVELOPMENT

Not applicable.

### BACKGROUND OF THE INVENTION

By the present application there are provided nonpeptidic scaffolds that serve as alpha-helix mimetics. More particularly, there are provided compounds, intermediates and methods for the preparation and uses thereof, and pharmaceutical compositions comprising nonpeptidic scaffolds having a pyridazine core.

α-Helices are the most common protein secondary structures and play a pivotal role in many protein-protein interactions. It is a rod-like structure wherein the polypeptide chain coils around like a corkscrew to form the inner part of the rod and the side chains extend outward in a helical array. Approximately 3.6 amino acid residues make up a single turn of an alpha-helix; thus the side chains that are adjacent in space and make up a “side” of an alpha-helix occur every three to four residues along the linear amino acid sequence. The alpha-helix conformation is stabilized by steric interactions along the backbone as well as hydrogen bonding interactions between the backbone amide carbonyls and NH groups of each amino acid. Frequently the critical interactions are found along a “face” of the helix involving side chains from the i, i+3 or i+4 and i+7 residues. These project from the α-helix with well known distances and angular relationships (Fairlie, D. P.; et al. Curr. Med. Chem. 1998, 5, 29-62; Jain, R.; et al. Mol. Divers. 2004, 8, 89-100; Cochran, A. G. Curr. Opin. Chem. Biol. 2001, 5, 654-659; Zutshi, R.; et al. Curr. Opin. Chem. Biol. 1998, 2, 62-66; Toogood, P. L. J. Med. Chem. 2002, 5, 1543-1558; Berg, T. Angew. Chem. Int. Ed. 2003, 42, 2462-2481).

Molecules that can predictably and selectively reproduce these projections could be valuable as tools in molecular biology and, potentially, as leads in drug discovery (Walensky, L. D.; et al. Science 2004, 305, 1466-1470). Nearly a third of the residues in known proteins form alpha-helices and such helices are important structural elements in various biological recognition events, including ligand-receptor interactions, protein-DNA interactions, protein-RNA interactions, and protein-membrane interactions. Given the importance of alpha-helices in biological systems, it would be desirable to have available small organic molecules that act as mimics of alpha-helices. Such compounds would be useful not only as research tools, but as therapeutics to treat conditions mediated by alpha-helix binding enzymes and receptors.

Side chains in positions i, i+3/i+4, i+7, and i+11 appear on the same face of the helix and are frequently crucial for the interaction (Davis, J. M.; et al. Chem. Soc. Rev. 2007, 36, 326; Fletcher, S.; Hamilton, A. D. J. R. Soc. Interface 2006, 3, 215; Yin, H.; Hamilton, A. D. Angew. Chem. Int. Ed. 2005, 44, 4130; Jain, R.; et al. Mol. Divers. 2004, 8, 89; Peczuh, M. W.; Hamilton, A. D. Chem. Rev. 2000, 100, 2479). Hamilton and co-workers pioneered the synthesis of non-peptidic α-helix mimetics based on terphenyl, terephthalamide, and oligopyridine scaffolds that display side chains in a manner that closely resembles those in position i, i+4, and i+7 of an α-helix (Kutzki, O.; et al. J. Am. Chem. Soc. 2002, 124, 11838; Ernst, J. T.; et al. Angew. Chem. Int. Ed. 2003, 42, 535; Yin, H.; et al. J. Am. Chem. Soc. 2005, 127, 5463). They were shown to efficiently disrupt protein-protein interactions such as Bak/Bcl-x_{L} (Yin, H.; et al. J. Am. Chem. Soc. 2005, 127, 10191), p53/HDM2 (Yin, H.; et al. Angew. Chem. Int. Ed. 2005, 44, 2704), calmodulin/smooth muscle myosin light-chain kinase (Orner, B. P.; et al. J. Am. Chem. Soc. 2001, 123, 5382), and gp41 assembly (Ernst, J. T.; et al. Angew. Chem. Int. Ed. 2002, 41, 278). During efforts towards the design of inhibitors of protein-protein interactions (Davis, C. N.; et al. Proc. Natl. Acad. Sci. USA 2006, 103, 2953; Bartfai, T.; et al. Proc. Natl. Acad. Sci. USA 2004, 101, 10470; Bartfai, T; et al. Proc. Natl. Acad. Sci. USA 2003, 100, 7971), methodology was developed for structurally similar molecules featuring more hydrophilic components and a facile synthetic route (Biros, S. M.; et al. Bioorg. Med. Chem. Lett. 2007, 17, 4641).

The syntheses of peptidomimetics having a stabilized α-helical conformation have been achieved by introducing synthetic templates into the peptidic chain (Kemp, D. S.; et al. J. Am. Chem. Soc. 1996, 118, 4240-4248; Austin, R. E.; et al. J. Am. Chem. Soc. 1997, 119, 6461-6472), by using β-hairpin mimetics (Fasan, R.; et al. Angew. Chem. Int. Ed. 2004, 43, 2109-2112), β-peptide sequences (Kritzer, J. A.; et al. J. Am. Chem. Soc. 2004, 126, 9468-9469), and unnatural oligomers with discrete folding propensities (foldamers) (Sadowsky, J. D.; et al. J. Am. Chem. Soc. 2005, 127, 11966-11968). Small synthetic molecules able to mimic the surfaces of constrained peptides offer the advantage of improved stability, lower molecular weight and in some cases better bioavailability. Although synthetic small molecules that adopt various well-defined secondary structures are well-documented (Hagihara, M.; et al. J. Am. Chem. Soc. 1992, 114, 6568-6570; Gennari, G.; et al. Angew. Chem. Int. Ed. Engl. 1994, 33, 2067-2069; Gude, M.; et al. Tetrahedron Lett. 1996, 37, 8589-8592; Cho, C. Y.; et al. Science 1993, 261, 1303-1305; Hamuro, Y.; et al. J. Am. Chem. Soc. 1996, 118, 7529-7541; Nowick, J. S.; et al. J. Am. Chem. Soc. 1996, 118, 1066-1072; Lokey, R. S.; Iverson, B. L. Nature, 1995, 375, 303-305; Murray, T. J.; Zimmerman S. C. J. Am. Chem. Soc. 1992, 114, 4010-4011; Antuch, W.; et al. Bioorg. Med. Chem. Lett. 2006, 16, 1740-1743. For reviews concerning α-helix mimetics, see: Yin, H.; Hamilton, A. D. Angew. Chem. Int. Ed. 2005, 44, 4130-4163; Fletcher, S.; Hamilton, A. D. J. R. Soc. Interface 2006, 3, 215-233; Davis, J. M.; et al. Chem. Soc. Rev. 2007, 36, 326-334. See also: Cummins, M. D.; et al. Chem. Biol. Drug Des. 2006, 67, 201-205; Ahn, J-M. Han, S-Y. Tetrahedron Lett. 2007, 48, 3543-3547), the first useful mimetics for an α-helix were reported only recently by Hamilton and coworkers: the terphenyl scaffold (Orner, B. P.; et al. J. Am. Chem. Soc. 2001, 123, 5382-5383; Yin, H.; et al. J. Am. Chem. Soc. 2005, 127, 10191-10196; Yin, H.; et al. Angew. Chem. Int. Ed. 2005, 44, 2704-2707), and its pyridine (Ernst, J. T.; et al. Angew. Chem. Int. Ed. 2003, 42, 535-539) and terephthalic acid (Yin, H.; Hamilton, A. D. Bioorg. Med. Chem. Lett. 2004, 14, 1375-1379) analogues.

Bak and Bcl-x_{L} belong to the Bcl-2 family of proteins, which regulate cell death through an intricate balance of homodimer and heterodimer complexes formed within this class of proteins (M. C. Raff, Science 1994, 264, 668-669; D. T. Chao, S. J. Korsmeyer, Annu. Rev. Immunol. 1998, 16, 395-419; C. B. Thompson, Science 1995, 267, 1456-1462; L. L. Rubin, K. L. Philpott, S. F. Brooks, Curr. Biol. 1993, 3, 391-394). Overexpression of anti-apoptotic proteins such as Bcl-x_{L} and Bcl-2 prevent cells from triggering programmed death pathways and has been linked to a variety of cancers. Bcl-2 protein plays a critical role in inhibiting anticancer drug-induced apoptosis, which is mediated by a mitochondria-dependent pathway that controls the release of cytochrome c from mitochondria through anion channels. Constitutive overexpression of Bcl-2 or unchanged expression after treatment with anticancer drugs confers drug resistance not only to hematologic malignancies but also to solid tumors (R. Kim et al. Cancer 2004, 101, 2491-2502). A current strategy for developing new anticancer agents is to identify molecules that bind to the Bak-recognition site on Bcl-x_{L}, disrupting the complexation of the two proteins and therefore antagonizing Bcl-x_{L} function (O. Kutzki et al. J. Am. Chem. Soc. 2002, 124, 11, 832-11, 839). The structure determined by NMR spectroscopy (M. Sattler et al. Science 1997, 275, 983-986) shows the 16 residue BH3 domain peptide from Bak (aa 72 to 87, K_{d}≈300 nM) bound in a helical conformation to a hydrophobic cleft on the surface of Bcl-x_{L}, formed by the BH1, BH2, and BH3 domains of the protein. The crucial residues for binding were shown by alanine scanning to be V74, L78, I81, and 185, which project in an i, i+4, i+7, i+11 arrangement from one face of the α-helix. The Bak peptide is a random coil in solution but adopts an α-helical conformation when complexed to Bcl-x_{L}. Studies utilizing stabilized helices of the Bak BH3 domain have shown the importance of this conformation for tight binding. (J. W. Chin, A. Schepartz, Angew. Chem. 2001, 113, 3922-3925; Angew. Chem. Int. Ed. 2001, 40, 3806-3809).

In connection with prior studies on the synthesis of heterocyclic α-helix mimetics (Biros, S. M.; et al. Bioorg. Med. Chem. Lett. 2007, 17, 4641-4645; Volonterio, A.; Moisan, L.; Rebek, J. Jr. Org. Lett. 2007, 9, 3733-3736; Moisan, L.; et al. Heterocycles 2007, prepress COM-07-S(U)45), the preparation of the 3,4,6-trisubstituted pyridazine 2a′, bearing an indole side chain was required. This structure is inspired by Hamilton's terephthalamide scaffold 3 known to disrupt protein-protein interactions (Yin, H.; Hamilton, A. D. Bioorg. Med. Chem. Lett. 2004, 14, 1375-1379; Yin, H.; et al. J. Am. Chem. Soc. 2005, 127, 5463-5468) when R^{1′}, R^{2′} and R^{3′} are typically side chains of hydrophobic amino acids. The pyridazine scaffold offers remote hydrophilic sites, regioselective functionalization (Biros, S. M.; et al. Bioorg. Med. Chem. Lett. 2007, 17, 4641-4645; Volonterio, A.; Moisan, L.; Rebek, J. Jr. Org. Lett. 2007, 9, 3733-3736; Moisan, L.; et al. Heterocycles 2007, prepress COM-07-S(U)45) and a variety of amino acid side chains for small library synthesis. Further pyridazine based alpha-helix mimetics having a variety of functional groups are disclosed in U.S. Provisional Patent application Ser. No. 60/965,100, filed Aug. 18, 2007, and incorporated herein by reference in its entirety and for all purposes. See structures for Cmpds 2a′ and 3 following.

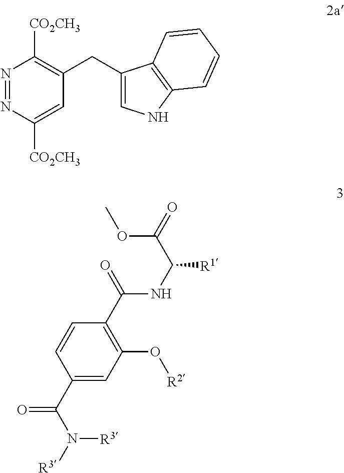

In addition to the above pyridazine based alpha-helix mimetics, the pyridazine ring is also encountered as a structural component of other compounds possessing biological activity: analgesic (Rohet, F.; et al. Bioorg. Med. Chem. 1997, 5, 655-659), antibacterial (Tucker, J. A.; et al. J. Med. Chem. 1998, 41, 3727-3735), antiinflammatory (Tamayo, N.; et al. Bioorg. Med. Chem. Lett. 2005, 15, 2409-2413), antihypertensive (Benson, S. C.; et al. J. Org. Chem. 1987, 52, 4610-4614) or antihistaminic (Gyoten, M.; et al. Chem. Pharm. Bull. 2003, 51, 122-133) activities have all been reported. This heterocycle is also useful for the preparation of other heterocycles (Naud, S.; et al. Eur. J. Org. Chem. 2007, 3296-3310), π-conjugated organic materials with desirable electronic properties (Yasuda, T.; et al. Chem. Mater. 2005, 17, 6060-6068) and self-assembled supramolecular architectures (Cuccia, L. A.; et al. Angew. Chem. Int. Ed. 2000, 39, 233-237). These pharmacological and technological properties of pyridazines encourage the development of methods for their synthesis and functionalization (Nara, S.; et al. Synlett 2006, 3185-3204). In this context, the Inverse Electron Demand Diels-Alder Reaction (IEDDAR) between 1,2,4,5-tetrazine diester 1 and electron-rich dienophiles has proven to be an effective synthetic route toward substituted pyridazines (Hamasaki, A.; et al. J. Org. Chem. 2006, 71, 185-193; Helm, M. D.; et al. Angew. Chem. Int. Ed. 2005, 44, 3889-3892). See FIG. 1.

Small molecule mimetics of alpha-helices are of immense pharmaceutical interest and would circumvent the problems associated with the use of peptidic agents. Accordingly, there is a need in the art for further small molecule compounds that can modulate the activity of alpha-helix mediated interactions and therefore would be useful in the treatment of a variety of diseases mediated by these proteins.

Disclosed herein is a novel class of low-molecular-weight α-helix mimetics featuring a pyridazine ring linked to a variety of amino acid side chains, and analogs thereof, which include hydrophobic amino-acid side chains.

### BRIEF SUMMARY OF THE INVENTION

In a first aspect, there is provided a compound having the structure of Formula (I).

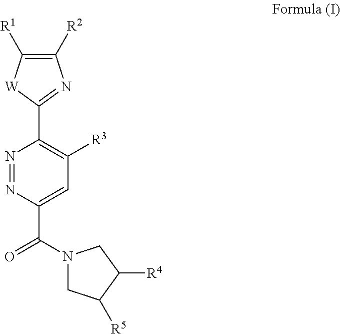

In Formula (I), W is —O— or —S—. R¹ and R² are independently hydrogen, substituted or unsubstituted alkyl, substituted or unsubstituted heteroalkyl, or a side chain of a naturally occurring amino acid, homolog thereof, or chemically protected analog thereof, optionally linked through an —O— ether linkage, provided that at least one of R¹ and R² is a side chain of a naturally occurring amino acid, homolog thereof, or chemically protected analog thereof, optionally linked through an —O— ether linkage. R³ is hydrogen, substituted or unsubstituted alkyl, or a side chain of a naturally occurring amino acid, homolog thereof, or chemically protected analog thereof. R⁴ and R⁵ are independently hydrogen, substituted or unsubstituted alkyl, substituted or unsubstituted heteroalkyl, or a side chain of a naturally occurring amino acid, homolog thereof, or chemically protected analog thereof, optionally linked through an —O— ether linkage, provided that at least one of R⁴ and R⁵ is a side chain of a naturally occurring amino acid, homolog thereof, or chemically protected analog thereof, optionally linked through an —O— ether linkage.

Another aspect of the invention is directed to a process for synthesizing any of the compounds of Formula (I) and intermediates thereof.

In another aspect, there is provided a method for disrupting a protein-protein interaction. The proteins involved in this interaction are selected from the group consisting of Bak/Bcl-x_{L}, p53/HDM2, calmodulin/smooth muscle myosin light-chain kinase, and the gp41 assembly. The method includes the step of contacting the proteins involved in the protein-protein interaction with a compound of Formula (I) with sufficient concentration to disrupt the protein-protein interaction. In a preferred mode, the process for treating conditions and/or disorders mediated by the disruption of the protein-protein interaction includes the step of administering a sufficient amount to a compound of Formula (I) to a patient to lead to the disruption of the protein-protein interaction.

### BRIEF DESCRIPTION OF THE DRAWINGS

FIG. 1 illustrates a two-reaction scheme with reactant ester providing the “R” group, wherein the R substituent ends up in pyridazine compound 2.

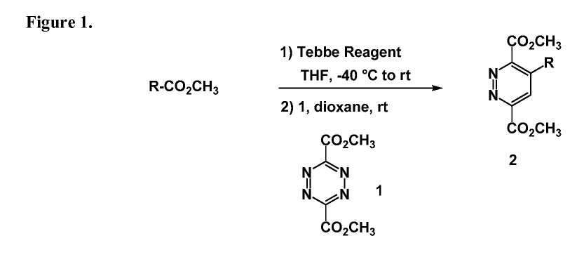

### DETAILED DESCRIPTION OF THE INVENTION

### I. Definitions

The following definitions are used throughout this specification.

Generally, reference to a certain element such as hydrogen or H is meant to include all isotopes of that element. For example, if an R group is defined to include hydrogen or H, it also includes deuterium and tritium.

Alkyl groups include straight chain and branched alkyl groups having from 1 to about 20 carbon atoms, and typically from 1 to 12 carbons or, in some embodiments, from 1 to 8 carbon atoms. As employed herein, “alkyl groups” include cycloalkyl groups as defined below. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, sec-butyl, t-butyl, and isopentyl groups. Representative substituted alkyl groups may be substituted one or more times with, for example, amino, carboxy, carboxamido, thio, hydroxy, alkoxy, and/or halo groups such as F, Cl, Br, and I groups.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which may be substituted with, for example, alkyl, alkoxy, amino, thio, hydroxy, cyano, and/or halo groups.

Cycloalkyl alkyl groups are alkyl groups as defined above in which a hydrogen or carbon bond of an alkyl group is replaced with a bond to a cycloalkyl group as defined above.

Alkenyl groups are straight chain, branched or cyclic alkyl groups having 2 to about 20 carbon atoms, and further including at least one double bond. In some embodiments alkenyl groups have from 2 to 12 carbons, or, typically, from 2 to 8 carbon atoms. Alkenyl groups include, for instance, vinyl, propenyl, 2-butenyl, 3-butenyl, isobutenyl, cyclohexenyl, cyclopentenyl, cyclohexadienyl, butadienyl, pentadienyl, and hexadienyl groups among others.

Alkynyl groups are straight chain or branched alkyl groups having 2 to about 20 carbon atoms, and further including at least one triple bond. In some embodiments alkynyl groups have from 2 to 12 carbons, or, typically, from 2 to 8 carbon atoms. Exemplary alkynyl groups include, but are not limited to, ethynyl, propynyl, and butynyl groups.

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH—₂—CH₃, and —CN. Up to two heteroatoms may be consecutive, such as, for example, —CH₂—NH—OCH₃.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

Aryl groups are cyclic aromatic hydrocarbons that do not contain heteroatoms. Thus, aryl groups include, but are not limited to, phenyl, azulenyl, heptalenyl, biphenylenyl, indacenyl, phenanthrenyl, triphenylenyl, pyrenyl, naphthacenyl, chrysenyl, biphenyl, anthracenyl, and naphthenyl groups. Although the phrase “aryl groups” includes groups containing fused rings, such as fused aromatic-aliphatic ring systems (e.g., indanyl, tetrahydronaphthyl, and the like) and fused aromatic-unsaturated ring systems (e.g., indenyl, fluorenyl, and the like). It does not include aryl groups that have other groups, such as alkyl or halo groups, bonded to one of the ring members. Rather, groups such as tolyl are referred to as substituted aryl groups. Representative substituted aryl groups may be mono-substituted or substituted more than once, such as, but not limited to, 2-, 3-, 4-, 5-, or 6-substituted phenyl or naphthyl groups, which may be substituted with groups including, but not limited to, amino, nitro, carboxy, carboxamido, hydroxy, thio, alkoxy, alkyl, cyano, and/or halo.

Aralkyl groups are alkyl groups as defined above in which a hydrogen or carbon bond of an alkyl group is replaced with a bond to an aryl group as defined above. Representative aralkyl groups include benzyl groups and fused (cycloalkylaryl)alkyl groups such as 4-ethyl-indanyl.

Heterocyclyl groups include aromatic and non-aromatic ring compounds containing 3 or more ring members, of which, one or more is a heteroatom such as, but not limited to, N, O, and S. In some embodiments, heterocyclyl groups include 3 to 20 ring members, whereas other such groups have 3 to 15 ring members. The phrase “heterocyclyl group” includes mono-, bi-, and polycyclic ring systems. Heterocyclyl groups thus include fused ring species including those comprising fused aromatic and non-aromatic groups. The phrase also includes bridged polycyclic ring systems containing one or more heteroatoms such as, but not limited to, quinuclidyl. However, the phrase does not include heterocyclyl groups that have other groups, such as alkyl or halo groups, bonded to one of the ring members. Rather, these are referred to as “substituted heterocyclyl groups”. Heterocyclyl groups include, but are not limited to, pyrrolidinyl, piperidinyl, piperazinyl, morpholinyl, pyrrolyl, pyrazolyl, triazolyl, imidazolyl, imidazolidinyl, tetrazolyl, oxazolyl, oxazolinyl, oxazolidinyl, isoxazolyl, isoxazolinyl, isoxazolidinyl, thiazolyl, pyridinyl, pyridazinyl, pyrazinyl, pyrimidinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups may be mono-substituted or substituted more than once, such as, but not limited to, pyridazinyl, pyridinyl, oxazolidinyl, oxazolinyl, or oxazolyl groups, which are 2-, 3-, 4-, 5-, or 6-substituted, or disubstituted with groups including, but not limited to, amino, hydroxyl, thio, alkoxy, alkyl, cyano, and/or halo.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, imidazolyl, triazolyl, tetrazolyl, oxazolyl, oxazolinyl, oxazolidinyl, isoxazolyl, isoxazolinyl, isoxazolidinyl, thiazolyl, pyridinyl, pyridazinyl, pyrazinyl, pyrimidinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Although the phrase “heteroaryl groups” includes fused ring compounds such as indolyl and 2,3-dihydroindolyl, the phrase does not include heteroaryl groups that have other groups bonded to one of the ring members, such as alkyl groups. Rather, heteroaryl groups with such substitution are referred to as “substituted heteroaryl groups”. Representative substituted heteroaryl groups may be substituted one or more times with groups including, but not limited to, amino, alkoxy, alkyl, thio, hydroxy, cyano, and/or halo.

Heterocyclylalkyl groups are alkyl groups as defined above in which a hydrogen or carbon bond of an alkyl group is replaced with a bond to a heterocyclyl group as defined above. Representative heterocyclyl alkyl groups include, but are not limited to, furan-2-yl methyl, furan-3-yl methyl, pyridin-3-yl methyl, tetrahydrofuran-2-yl ethyl, indol-2-yl methyl, and indol-2-yl propyl.

Heteroaralkyl groups are alkyl groups as defined above in which a hydrogen or carbon bond of an alkyl group is replaced with a bond to a heteroaryl group as defined above.

In general, “substituted” refers to a group as defined above in which one or more bonds to a hydrogen atom contained therein are replaced by a bond to non-hydrogen atoms such as, but not limited to, a halogen atom such as F, Cl, Br, and I; an oxygen atom in groups such as hydroxyl groups, alkoxy groups, aryloxy groups, aralkoxy groups, and ester groups; a sulfur atom in groups such as thiol groups, alkyl and aryl sulfide groups, sulfone groups, sulfonyl groups, sulfonamide, and sulfoxide groups; a nitrogen atom in groups such as nitro groups, amines, amides, alkylamines, dialkylamines, arylamines, alkylarylamines, diarylamines, N-oxides, imides, ureas, guanidines, amidines and enamines; a silicon atom in groups such as in trialkylsilyl groups, dialkylarylsilyl groups, alkyldiarylsilyl groups, and triarylsilyl groups; and other heteroatoms in various other groups. Substituted alkyl groups and also substituted cycloalkyl groups and others also include groups in which one or more bonds to a carbon(s) or hydrogen(s) atom is replaced by a bond to a heteroatom such as oxygen in carbonyl, carboxyl, and ester groups; nitrogen in groups such as imines, oximes, hydrazones, and nitriles.

Substituted ring systems such as, but not limited to, substituted cycloalkyl, aryl, heterocyclyl and heteroaryl groups also include rings and fused ring systems in which a bond to a hydrogen atom is replaced with a bond to a carbon atom. Therefore, substituted cycloalkyl, aryl, heterocyclyl and heteroaryl groups may also be substituted with alkyl groups, alkenyl groups, or alkynyl groups as defined above.

The term “protected” with respect to hydroxyl groups, amine groups, and sulfhydryl groups refers to forms of these functionalities which are protected from undesirable reaction with a protecting group known to those skilled in the art such as those set forth in Protective Groups in Organic Synthesis, Greene, T. W.; Wuts, P. G. M., John Wiley & Sons, New York, N.Y., (3rd Edition, 1999) which can be added or removed using the procedures set forth therein. Examples of protected hydroxyl groups include, but are not limited to, silyl ethers such as those obtained by reaction of a hydroxyl group with a reagent such as, but not limited to, t-butyldimethylchlorosilane, trimethylchlorosilane, triisopropylchlorosilane, triethylchlorosilane; substituted methyl and ethyl ethers such as, but not limited to methoxymethyl ether, methylthiomethyl ether, benzyloxymethyl ether, t-butoxymethyl ether, 2-methoxyethoxymethyl ether, tetrahydropyranyl ethers, 1-ethoxyethyl ether, allyl ether, benzyl ether; esters such as, but not limited to, benzoylformate, formate, acetate, trichloroacetate, and trifluoracetate. Examples of protected amine groups include, but are not limited to, amides such as, formamide, acetamide, trifluoroacetamide, and benzamide; imides, such as phthalimide, and dithiosuccinimide; and others. Examples of protected sulfhydryl groups include, but are not limited to, thioethers such as S-benzyl thioether, and S-4-picolyl thioether; substituted S-methyl derivatives such as hemithio, dithio and aminothio acetals; and others. A “chemically protected analog,” as used herein, refers to a protected a compound described herein that is protected. The “chemically protected analog” may have one or a plurality of protecting groups.

Side chains of amino acids are the groups attached to the alpha carbon of alpha-amino acids. For example the side chains of glycine, alanine, and phenylalanine are hydrogen, methyl, and benzyl, respectively. The side chains may be of any naturally occurring or synthetic alpha amino acid. Naturally occurring alpha amino acids include those found in naturally occurring peptides, proteins, hormones, neurotransmitters, and other naturally occurring molecules. Synthetic alpha amino acids include any non-naturally occurring amino acid known to those of skill in the art. Representative amino acids include, but are not limited to, glycine, alanine, serine, threonine, arginine, lysine, ornithine, aspartic acid, glutamic acid, asparagine, glutamine, phenylalanine, tyrosine, tryptophan, leucine, valine, isoleucine, cysteine, methionine, histidine, 4-trifluoromethyl-phenylalanine, 3-(2-pyridyl)-alanine, 3-(2-furyl)-alanine, 2,4-diaminobutyric acid, and the like.

Pharmaceutically acceptable salts include a salt with an inorganic base, organic base, inorganic acid, organic acid, or basic or acidic amino acid. As salts of inorganic bases, the invention includes, for example, alkali metals such as sodium or potassium, alkali earth metals such as calcium and magnesium or aluminum, and ammonia. As salts of organic bases, the invention includes, for example, trimethylamine, triethylamine, pyridine, picoline, ethanolamine, diethanolamine, triethanolamine. As salts of inorganic acids, the instant invention includes, for example, hydrochloric acid, boric acid, nitric acid, sulfuric acid, and phosphoric acid. As salts of organic acids, the instant invention includes, for example, formic acid, acetic acid, trifluoroacetic acid, fumaric acid, oxalic acid, tartaric acid, lactic acid, maleic acid, citric acid, succinic acid, malic acid, methanesulfonic acid, benzenesulfonic acid, and p-toluenesulfonic acid. As salts of basic amino acids, the instant invention includes, for example, arginine, lysine and ornithine. Acidic amino acids include, for example, aspartic acid and glutamic acid.

Certain compounds within the scope of Formula I and Formula IA are derivatives referred to as prodrugs. The expression “prodrug” denotes a derivative of a known direct acting drug, e.g. esters and amides, which derivative has enhanced delivery characteristics and therapeutic value as compared to the drug, and is transformed into the active drug by an enzymatic or chemical process; see Notari, R. E., “Theory and Practice of Prodrug Kinetics,” Methods in Enzymology 112:309-323 (1985); Bodor, N., “Novel Approaches in Prodrug Design,” Drugs of the Future 6:165-182 (1981); and Bundgaard, H., “Design of Prodrugs: Bioreversible-Derivatives for Various Functional Groups and Chemical Entities,” in Design of Prodrugs (H. Bundgaard, ed.), Elsevier, New York (1985), Goodman and Gilmans, The Pharmacological Basis of Therapeutics, 8th ed., McGraw-Hill, Int. Ed. 1992. The preceding references are hereby incorporated by reference in their entirety and for all purposes.

Tautomers refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, ketones are typically in equilibrium with their enol forms. Thus, ketones and their enols are referred to as tautomers of each other. As readily understood by one skilled in the art, a wide variety of functional groups and other structures may exhibit tautomerism, and all tautomers of compounds having Formula I or Formula IA are within the scope of the present invention.

Compounds of the present invention include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these are all within the scope of the invention.

“Treating” within the context of the instant invention, means an alleviation, in whole or in part, of symptoms associated with a disorder or disease, or halt of further progression or worsening of those symptoms, or prevention or prophylaxis of the disease or disorder. Similarly, as used herein, a “therapeutically effective amount” of a compound of the invention refers to an amount of the compound that alleviates, in whole or in part, symptoms associated with a disorder or disease, or halts of further progression or worsening of those symptoms, or prevents or provides prophylaxis for the disease or disorder. Treatment may also include administering the pharmaceutical Formulations of the present invention in combination with other therapies. For example, the compounds of the invention can also be administered in conjunction with other therapeutic agents against bone disease or agents used for the treatment of metabolic disorders.

### II. Compositions

In one aspect, there is provided a compound having the structure of Formula (I). Compounds having the structure of Formula (I) may be nonpeptidic mimetics of a peptide alpha-helix.

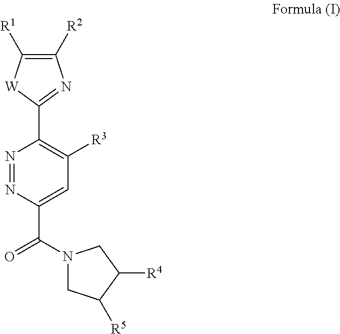

In Formula (I), W is —O— or —S—. R¹ and R² are independently hydrogen, substituted or unsubstituted alkyl, substituted or unsubstituted heteroalkyl, or a side chain of a naturally occurring amino acid, homolog thereof, or chemically protected analog thereof, optionally linked through an —O— ether linkage, provided that at least one of R¹ and R² is a side chain of a naturally occurring amino acid, homolog thereof, or chemically protected analog thereof, optionally linked through an —O— ether linkage. R³ is hydrogen, substituted or unsubstituted alkyl, or a side chain of a naturally occurring amino acid, homolog thereof, or chemically protected analog thereof. R⁴ and R⁵ are independently hydrogen, substituted or unsubstituted alkyl, substituted or unsubstituted heteroalkyl, or a side chain of a naturally occurring amino acid, homolog thereof, or chemically protected analog thereof, optionally linked through an —O— ether linkage, provided that at least one of R⁴ and R⁵ is a side chain of a naturally occurring amino acid, homolog thereof, or chemically protected analog thereof, optionally linked through an —O— ether linkage. “Homolog” refers in the customary sense to elongation (or shortening) of a substituent described herein by insertion (or deletion) of one or more hydrocarbon functionalities. For example, diaminopropionic acid, diaminobutyric acid, ornithine, lysine, and homolysine form a homologous series for lysine. The term “chemically protected analog” refers to compounds having chemical protecting groups. Exemplary chemical protecting groups are well known in the art and include, but not limited to, Boc, FMoc, benzyl (Bn), tert-Bu, trityl (—CPh₃), and the like. Where R¹, R², R⁴ and/or R⁵ are “optionally linked through an —O— ether linkage”, it is meant that R¹, R², R⁴ and/or R⁵ is indirectly attached to the remained of the molecule via a divalent oxygen linker (i.e. an ether).

Thus, in some embodiments, R¹, R², R⁴ and/or R⁵ are -L¹-R^{1A}, -L²-R^{2A}, -L⁴-R^{4A} and/or -L⁵-R^{5A}, respectively. R^{1A}, R^{2A}, R^{4A} and/or R^{5A} are independently hydrogen, substituted or unsubstituted alkyl, substituted or unsubstituted heteroalkyl, or a side chain of a naturally occurring amino acid, homolog thereof, or chemically protected analog thereof, provided that at least one of R^{1A} and R^{2A} is a side chain of a naturally occurring amino acid, homolog thereof, or chemically protected analog thereof. L¹, L², L⁴ and L⁵ are independently a bond or —O—. In some embodiments, the definitions of R¹, R², R⁴ and/or R⁵ described herein (without regard to the optionally linkage through an ether linkage) are equally applicable to R^{1A}, R^{2A}, R^{4A} and/or R^{5A}, respectively. Thus, in some embodiments, the compound has the formula:

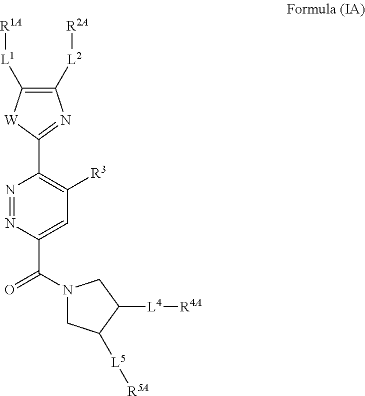

In Formula (IA), R^{1A}, R^{2A}, R^{4A} and R^{5A} are, respectively, defined the same as R¹, R², R⁴ and R⁵ as described herein (without regard to the portions of the R¹, R², R⁴ and R⁵ definitions relating to the optional ether linkage). L¹, L², L⁴ and L⁵ are independently a bond or —O—. R³ and W are as defined herein.

Without wishing to be bound by any theory, it is believed that compounds of Formula (I) and/or (IA) useful in the methods described herein mimic at least part of the structure of an alpha-helical segment of a protein involved in a protein-protein interaction. Accordingly, either of substituents R¹ and R² may mimic the side chain of a residue protruding away from the backbone of an alpha helical segment at an arbitrary residue (with index “i”) within the protein sequence, R³ may mimic the side chain of residue i+3 or i+4 within the sequence, and/or either of substituents R⁴ and R⁵ may mimic the side chain of residue i+7 within the sequence. The term “i+x” in the context of proteins refers, in the customary sense, to a residue having a position in the primary sequence of the protein which is “x” residues beyond a residue “i.” Alternatively, either of substituents R⁴ and R⁵ may mimic the side chain of a residue protruding away from the backbone of an alpha helical segment at a residue “i” within the protein sequence, R³ may mimic the side chain of residue i+3 or i+4 within the sequence, and/or either of substituents R¹ and R² may mimic the side chain of residue i+7 within the sequence. The terms “mimic the chain side of a residue” and the like in this context refer to a conformation of a compound of Formula (I) wherein the distance between either of substituents R¹ or R² and R³, and the distance between R³ and either of substituents R⁴ or R⁵ are approximately the distances between the side chains of residues in an alpha-helix at the i to i+3/i+4, and i to i+7 positions, as known in the art.

In some embodiments, R¹, R², R³, R⁴ and R⁵ are as described above, with the proviso that, at most, only one of R¹ and R², R³, and R⁴ and R⁵ is hydrogen.

In some embodiments, R¹ and R² are independently hydrogen, substituted or unsubstituted alkyl, substituted or unsubstituted heteroalkyl, or a side chain of a naturally occurring amino acid, homolog thereof, or chemically protected analog thereof, provided that at least one of R¹ and R² is a side chain of a naturally occurring amino acid, homolog thereof, or chemically protected analog thereof, and R⁴ and R⁵ are independently hydrogen, substituted or unsubstituted alkyl, substituted or unsubstituted heteroalkyl, or a side chain of a naturally occurring amino acid, homolog thereof, or chemically protected analog thereof, provided that at least one of R⁴ and R⁵ is a side chain of a naturally occurring amino acid, homolog thereof, or chemically protected analog thereof.

In some embodiments, R¹, R^{1A}, R² and R^{2A} are independently hydrogen, R⁶-substituted or unsubstituted alkyl, R⁶-substituted or unsubstituted heteroalkyl, or a chemically protected analog thereof. R⁶ is independently halogen, —CN, —CF₃, —OH, —NH₂, —SO₂, —COOH, R⁷-substituted or unsubstituted alkyl, R⁷-substituted or unsubstituted heteroalkyl, R⁷-substituted or unsubstituted cycloalkyl, R⁷-substituted or unsubstituted heterocycloalkyl, R⁷-substituted or unsubstituted aryl, or R⁷-substituted or unsubstituted heteroaryl. R⁷ is independently halogen, —NO₂, —CN, —CF₃, —OH, —NH₂, —SO₂, —COOH, R⁸-substituted or unsubstituted alkyl, R⁸-substituted or unsubstituted heteroalkyl, R⁸-substituted or unsubstituted cycloalkyl, R⁸-substituted or unsubstituted heterocycloalkyl, R⁸-substituted or unsubstituted aryl, or R⁸-substituted or unsubstituted heteroaryl. R⁸ is independently halogen, —NO₂, —CN, —CF₃, —OH, —NH₂, —SO₂, —COOH, unsubstituted alkyl, unsubstituted heteroalkyl, unsubstituted cycloalkyl, unsubstituted heterocycloalkyl, unsubstituted aryl, or unsubstituted heteroaryl. In some embodiments, R¹, R^{1A}, R² and R^{2A} are independently hydrogen, R⁶-substituted or unsubstituted C₁-C₁₀ (e.g., C₁-C₆) alkyl, R⁶-substituted or unsubstituted 2 to 10 membered (e.g., 2 to 6 membered) heteroalkyl, or a chemically protected analog thereof.

In some embodiments, R¹ and R² are independently hydrogen, unsubstituted alkyl, unsubstituted heteroalkyl, or a side chain of a naturally occurring amino acid, homolog thereof, or chemically protected analog thereof, provided that at least one of R¹ and R² is a side chain of a naturally occurring amino acid, homolog thereof, or chemically protected analog thereof; and R⁴ and R⁵ are independently hydrogen, unsubstituted alkyl, unsubstituted heteroalkyl, or a side chain of a naturally occurring amino acid, homolog thereof, or chemically protected analog thereof, provided that at least one of R⁴ and R⁵ is a side chain of a naturally occurring amino acid, homolog thereof, or chemically protected analog thereof.

In some embodiments, R¹ and R² are independently hydrogen, substituted or unsubstituted alkyl, substituted or unsubstituted heteroalkyl, or a side chain of a naturally occurring amino acid, homolog thereof, or chemically protected analog thereof, and R⁴ and R⁵ are independently hydrogen, substituted or unsubstituted alkyl, substituted or unsubstituted heteroalkyl, or a side chain of a naturally occurring amino acid, homolog thereof, or chemically protected analog thereof.

In some embodiments, R³ is hydrogen, substituted or unsubstituted alkyl, or a side chain of a naturally occurring amino acid.

In some embodiments, R³ is hydrogen, R⁹-substituted or unsubstituted alkyl, or a chemically protected analog thereof. R⁹ is independently halogen, —CN, —NO₂, —CF₃, —OH, —NH₂, —SO₂, —COOH, R¹⁰-substituted or unsubstituted alkyl, R¹⁰-substituted or unsubstituted heteroalkyl, R¹⁰-substituted or unsubstituted cycloalkyl, R¹⁰-substituted or unsubstituted heterocycloalkyl, R¹⁰-substituted or unsubstituted aryl, or R¹⁰-substituted or unsubstituted heteroaryl. R¹⁰ is independently halogen, —NO₂, —CN, —CF₃, —OH, —NH₂, —SO₂, —COOH, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl. R¹¹ is independently halogen, —NO₂, —CN, —CF₃, —OH, —NH₂, —SO₂, —COOH, unsubstituted alkyl, unsubstituted heteroalkyl, unsubstituted cycloalkyl, unsubstituted heterocycloalkyl, unsubstituted aryl, or unsubstituted heteroaryl. In some embodiments, R³ is hydrogen, R⁹-substituted or unsubstituted C₁-C₁₀ (e.g., C₁-C₆) alkyl, or a chemically protected analog thereof.

In some embodiments, R³ is -(substituted or unsubstituted C₁-C₉ alkyl), —CH₂-(substituted or unsubstituted C₃-C₈ cycloalkyl), —CH₂-(substituted or unsubstituted C₆-C₁₀ aryl), —(CH₂)₂—SCPh₃, —(CH₂)₂—SMe, —(CH₂)₃—NBoc, —(CH₂)₂—NBoc,

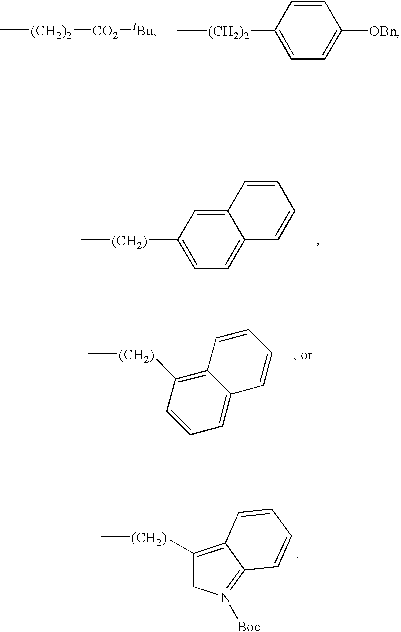

In other embodiments, R³ is -(unsubstituted C₁-C₉ alkyl), —CH₂-(unsubstituted C₃-C₈ cycloalkyl), —CH₂-(unsubstituted C₆-C₁₀ aryl), —(CH₂)₂—SCPh₃, —(CH₂)₂—SMe, —(CH₂)₃—NBoc, —(CH₂)₂—NBoc, —(CH₂)₂—CO₂-^{t}Bu,

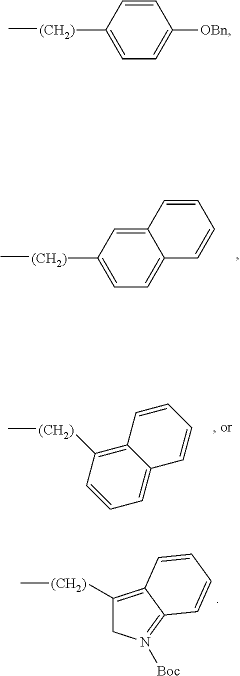

In some embodiments, R³ is selected from -(substituted or unsubstituted C₁-C₉ alkyl), —CH₂-(substituted or unsubstituted C₃-C₈ cycloalkyl), —CH₂-(substituted or unsubstituted C₆-C₁₀ aryl), —(CH₂)₂—SH, —(CH₂)₂—SMe, —(CH₂)₃—NH₂, —(CH₂)₂—NH₂,

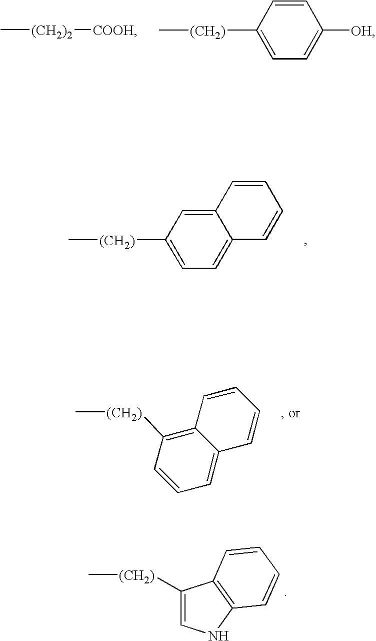

In other embodiments, R³ is selected from -(unsubstituted C₁-C₉ alkyl), —CH₂-(unsubstituted C₃-C₈ cycloalkyl), —CH₂-(unsubstituted C₆-C₁₀ aryl), —(CH₂)₂—SH, —(CH₂)₂—SMe, —(CH₂)₃—NH₂, —(CH₂)₂—NH₂, —(CH₂)₂—COOH,

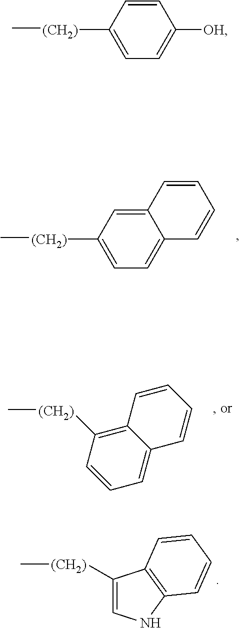

In some embodiments, R³ is selected from the group consisting of —(C₁-C₉ substituted or unsubstituted alkyl), substituted —CH₂-(substituted or unsubstituted C₃-C₈ cycloalkyl), —CH₂-(substituted or unsubstituted C₆-C₁₀ aryl), —(CH₂)₂—SCPh₃, —(CH₂)₂—SMe, —(CH₂)₃—NBoc, —(CH₂)₂—NBoc, and —(CH₂)₂—CO₂-^{t}Bu.

In some embodiments, R³ is selected from the group consisting of -(unsubstituted C₁-C₉ alkyl), —CH₂-(unsubstituted C₃-C₈ cycloalkyl), —CH₂-(unsubstituted C₆-C₁₀ aryl), —(CH₂)₂—SCPh₃, —(CH₂)₂—SMe, —(CH₂)₃—NBoc, —(CH₂)₂—NBoc, and —(CH₂)₂—CO₂-^{t}Bu.

In some embodiments, R⁴ and R⁵ are independently hydrogen, substituted or unsubstituted alkyl, substituted or unsubstituted heteroalkyl, or a side chain of a naturally occurring amino acid. In some embodiments, R⁴ and R⁵ are independently hydrogen, unsubstituted alkyl, unsubstituted heteroalkyl, or a side chain of a naturally occurring amino acid. In some embodiments, at least one of R⁴ and R⁵ is selected from the group consisting of —O-(substituted or unsubstituted C₁-C₆ alkyl), —O—(C₁-C₆ substituted or unsubstituted alkyl)-CO₂H, —O—C(O)-(substituted or unsubstituted C₁-C₆ alkyl), and —O—C(O)-(substituted or unsubstituted C₁-C₆ alkyl)-CO₂H. In some embodiments, at least one of R⁴ and R⁵ is selected from the group consisting of —O-(unsubstituted C₁-C₆ alkyl), —O-(unsubstituted C₁-C₆ alkyl)-CO₂H, —O—C(O)-(unsubstituted C₁-C₆ alkyl), and —O—C(O)-(unsubstituted C₁-C₆ alkyl)-CO₂H. In some embodiments, at least one of R⁴ and R⁵ is selected from the group consisting of -(substituted or unsubstituted C₁-C₆ alkyl), -(substituted or unsubstituted C₁-C₆ alkyl)-CO₂H, —C(O)-(substituted or unsubstituted C₁-C₆ alkyl), and —C(O)-(substituted or unsubstituted C₁-C₆ alkyl)-CO₂H. In some embodiments, at least one of R⁴ and R⁵ is selected from the group consisting of -(unsubstituted C₁-C₆ alkyl), -(unsubstituted C₁-C₆ alkyl)-CO₂H, —C(O)-(unsubstituted C₁-C₆ alkyl), and —C(O)-(unsubstituted C₁-C₆ alkyl)-CO₂H.

In some embodiments, R⁴, R^{4A}, R⁵ and R^{5A} are independently hydrogen, R¹²-substituted or unsubstituted alkyl, R¹²-substituted or unsubstituted heteroalkyl, or a chemically protected analog thereof. R¹² is independently halogen, —NO₂, —CN, —CF₃, —OH, —NH₂, —SO₂, —COOH, R¹³-substituted or unsubstituted alkyl, R¹³-substituted or unsubstituted heteroalkyl, R¹³-substituted or unsubstituted cycloalkyl, R¹³-substituted or unsubstituted heterocycloalkyl, R¹³-substituted or unsubstituted aryl, or R¹³-substituted or unsubstituted heteroaryl. R¹³ is independently halogen, —NO₂, —CN, —CF₃, —OH, —NH₂, —SO₂, —COOH, R¹⁴-substituted or unsubstituted alkyl, R¹⁴-substituted or unsubstituted heteroalkyl, R¹⁴-substituted or unsubstituted cycloalkyl, R¹⁴-substituted or unsubstituted heterocycloalkyl, R¹⁴-substituted or unsubstituted aryl, or R¹⁴-substituted or unsubstituted heteroaryl. R¹⁴ is independently halogen, —NO₂, —CN, —CF₃, —OH, —NH₂, —SO₂, —COOH, unsubstituted alkyl, unsubstituted heteroalkyl, unsubstituted cycloalkyl, unsubstituted heterocycloalkyl, unsubstituted aryl, or unsubstituted heteroaryl. In some embodiments, R⁴, R^{4A}, R⁵ and R^{5A} are independently hydrogen, R¹²-substituted or unsubstituted C₁-C₁₀ (e.g., C₁-C₆) alkyl, R¹²-substituted or unsubstituted 2 to 10 membered (e.g., 2 to 6 membered) heteroalkyl, or a chemically protected analog thereof.

In some embodiments, only one of R¹ and R² is hydrogen, and one of R⁴ and R⁵ is hydrogen. In some embodiments, only one of R¹ and R², R³, and R⁴ and R⁵ is hydrogen.

In some embodiments, the side chain of the naturally occurring amino acid forming substituent R¹, R², R³, R⁴, and/or R⁵ is a radical selected from the group of radicals consisting of —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, —O—(C₁-C₆ alkyl), and —OC(O)—(C₁-C₆ alkyl) and homologs thereof. In some embodiments, the side chain of the naturally occurring amino acid forming substituent R¹, R², R³, R⁴, and/or R⁵ is a radical selected from the group of radicals consisting of —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, —O—(C₁-C₆ alkyl), and —OC(O)—(C₁-C₆ alkyl). In some embodiments, the side chain of the naturally occurring amino acid forming substituent R¹, R², R³, R⁴, and/or R⁵ is a radical selected from the group of radicals consisting of —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, —O—(C₁-C₆ alkyl), and —OC(O)—(C₁-C₆ alkyl), and chemically protected analog thereof. In some embodiments, the side chain of the naturally occurring amino acid forming substituent R¹, R², R³, R⁴, and/or R⁵ is a radical selected from the group of radicals consisting of —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂-(4-imidazole), —CH₂CH₂CH₂NHC(NH)NH₂, —O—(C₁-C₆ alkyl), and —OC(O)-(substituted or unsubstituted C₁-C₆ alkyl) (e.g. OC(O)-(unsubstituted C₁-C₆ alkyl).

Exemplary compounds having the structure of Formula (I) are represented by the following structures:

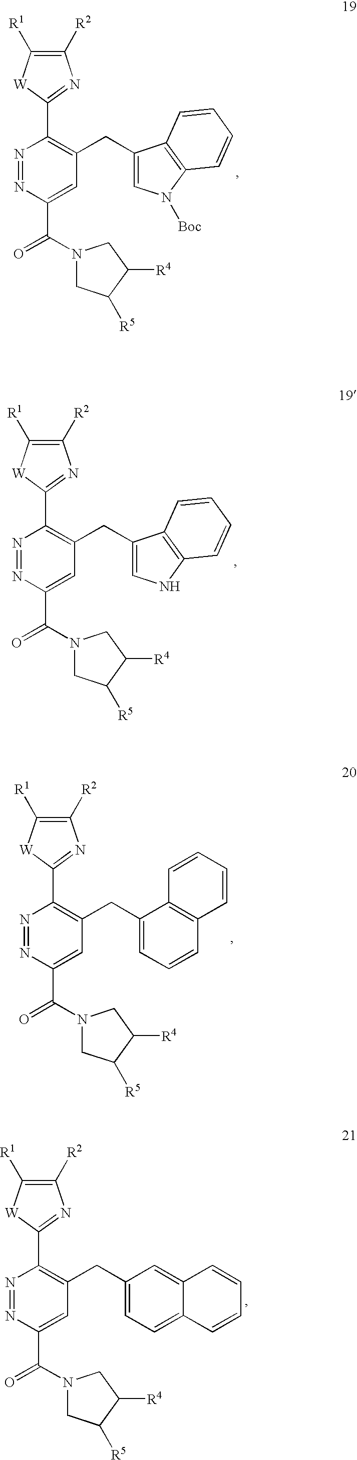

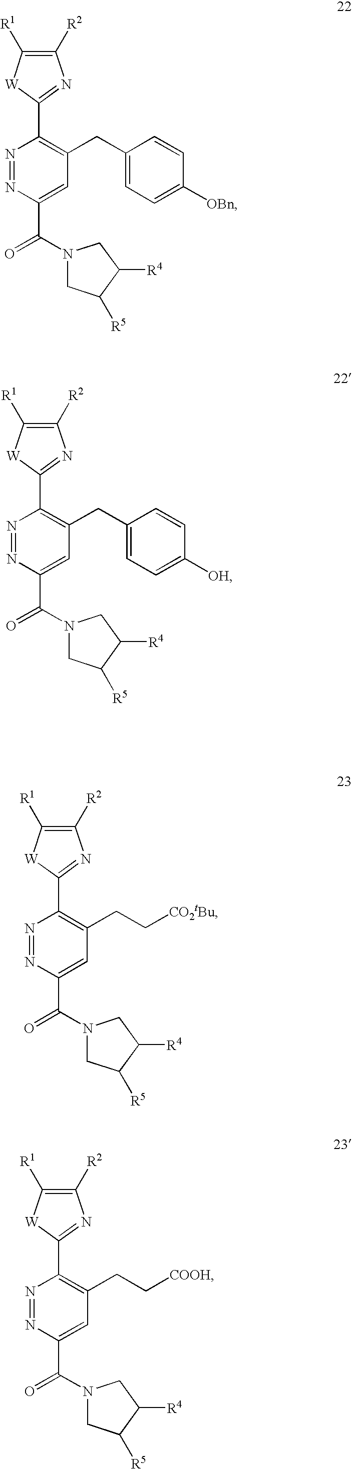

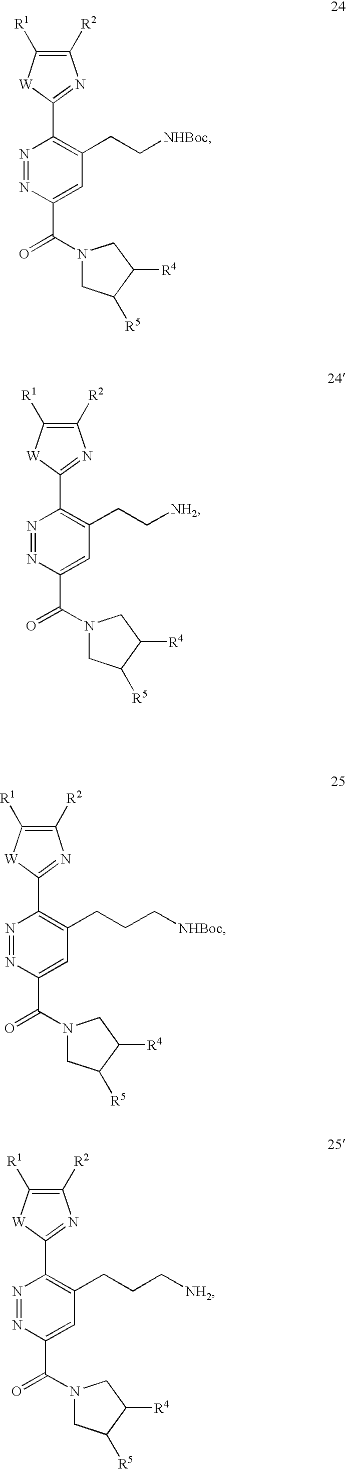

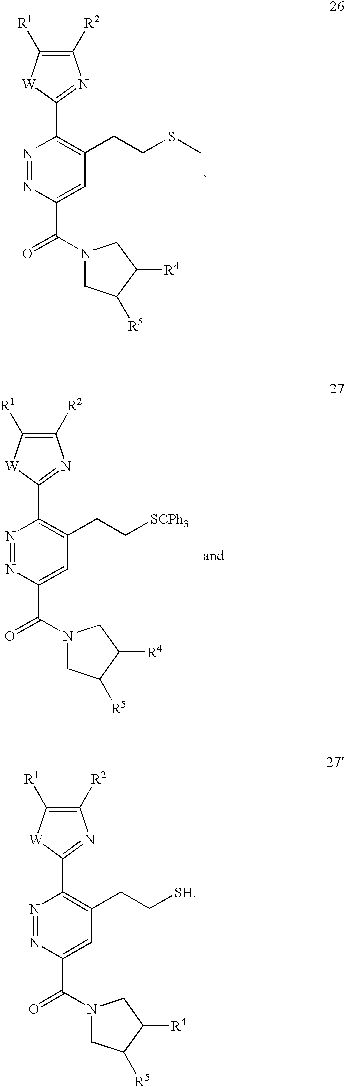

### III. Exemplary Syntheses

Methods for the preparation of compounds of Formula (I) and/or (IA) may proceed by an inverse electron demand Diels Alder reaction (IEDDAR) of a 1,2,4,5-tetrazine as the key step to forming the pyridazine structures. See Boger et al. J. Org. Chem. 1984 49, 4405; Boger, Tetrahedron 1983 39, 2869. Typically, the Diels Alder reaction is performed in an organic solvent such as diethyl ether, pentane, toluene, chloroform, dioxane, carbon tetrachloride, nitrobenzene, dichloromethane, ethyl acetate, THF, benzene, acetonitrile, dimethyl ether, 1,2-dichloroethane, xylene, acetone, chlorobenzene, DMSO, methanol, mesitylene and the like, or mixtures thereof. The reaction can be performed at room temperature, or can be heated to between 80-140° C. Typically, the reaction is performed at 80-100° C. Scheme 1 following illustrates a typical procedure to obtain intermediates to compounds of Formula I or Formula IA, wherein R¹, R² and R³ are as defined herein. The starting dimethyl 1,2,4,5-tetrazine-dicarboxylate is prepared by known methods. See Boger et al., Org. Synth. 1992, 70, 79; Spencer et al., J. Chem. Phys. 1961, 35, 1939; Sauer et al., Chem. Ber., 1965, 98, 1435.

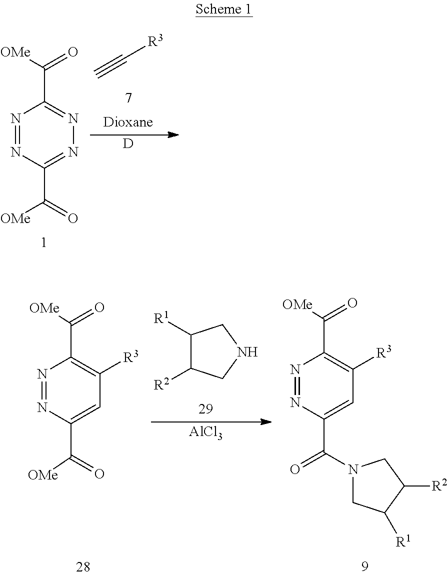

The inverse electron demand Diels Alder reaction with 1,2,4,5-tetrazine to form compounds of Formula I or Formula IA can also be performed with alkenes substituted with a leaving group, such as O-TMS, —SO-phenyl, morpholino, or pyrrolidino and the like (see Boger D. L. Tetrahedron 1983, Id.), by essentially the same procedures as described for Scheme 1.

Additionally, enolates, which can be generated in situ by reaction with aldehydes in the presence of a base (such as KOH, NaOH, LiOH, KOtBu, NaOMe, NaOEt, NaH and the like) can be used as reaction partners in the Diels Alder reaction. Scheme 2 shows an additional application, in which the Diels Alder reaction is performed with dihydrofuran or dihydropyran derivatives, yielding compounds useful in the synthesis of compounds of Formula I or Formula IA, wherein n is 1, 2 or 3.

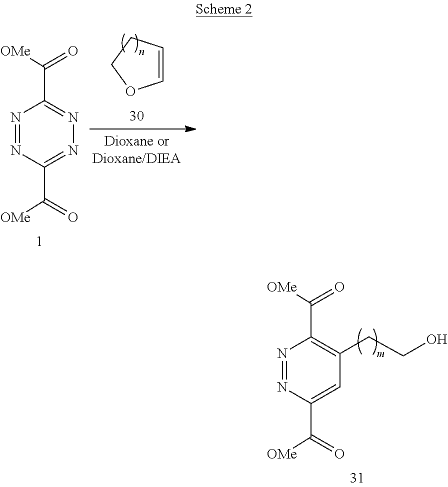

Further synthetic utility is available via the Diels Alder reaction of dihydrofuran, dihydropyran and substituted derivatives thereof with the tetrazine. For example, as shown in Scheme 3, the diester 1 (Boger et al. Org. Synth. 1992, Id.; Naud, Synlett 2004, 2836-2837) can be reacted with the commercially available 2-methoxy-3,4-dihydro-2H-pyrane to give pyridazine 4 in good yield. Subsequent treatment under standard Fischer indole synthesis (Hutchins & Chapman, Tetrahedron Lett. 1996, 37, 4869-4872) conditions leads predominantly to the decomposition of the starting material and formation of the desired unprotected compound 2a.

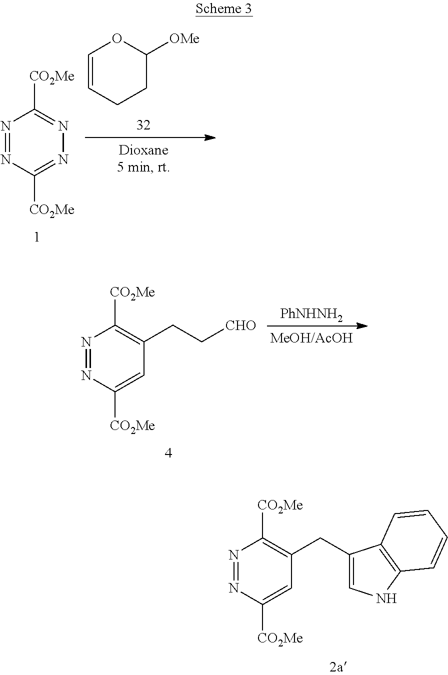

An additional synthetic route employs IEDDAR with a dienophile having an indole ring, or another substituent, already present. Different electron rich dienophiles such as enamines (Geyelin, P. H.; et al. ARKIVOC 2007, Part 11, 37-45), ketene acetals (Hartmann, K. P.; Heuschmann, M. Tetrahedron 2000, 56, 4213-4218), or enol ethers (Akiyama, T.; Morita, H.; Fuchibe, K. J. Am. Chem. Soc. 2006, 128, 13070-13071) have been employed in this type of [4+2]cycloaddition reaction. Since the conversion of esters into enol ethers by reaction with the Tebbe reagent is a well established procedure (Hartley, R. C.; McKiernan, G. J. J. Chem. Soc., Perkin Trans. 1 2002, 2763-2793), the N-Boc protected derivative 5 of the commercial available methyl-2-(1H-indol-3-yl)acetate was selected as the precursor of the electron rich dienophile 6. See Scheme 4 following. Treatment of compound 5 with the Tebbe reagent in tetrahydrofuran at low temperature can afford the desired enol ether 6 Subsequent reaction with tetrazine 1 at room temperature can afford pyridazine 2a. Cmpd 2a′ is obtainable from cmpd 2a by deprotection of the protecting Boc group by methods well known in the art.

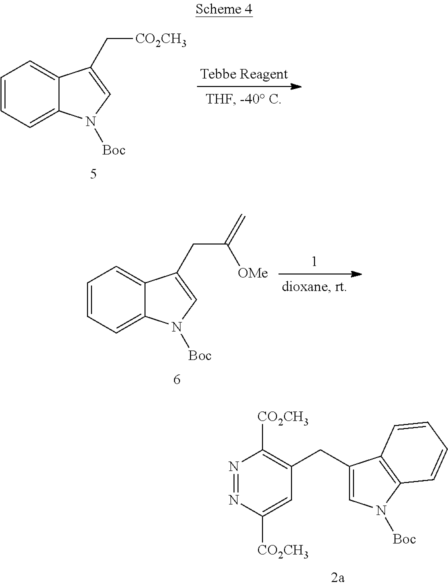

A variety of methods are available for further elaboration of the compound resulting from Scheme 1. See Scheme 5 following.

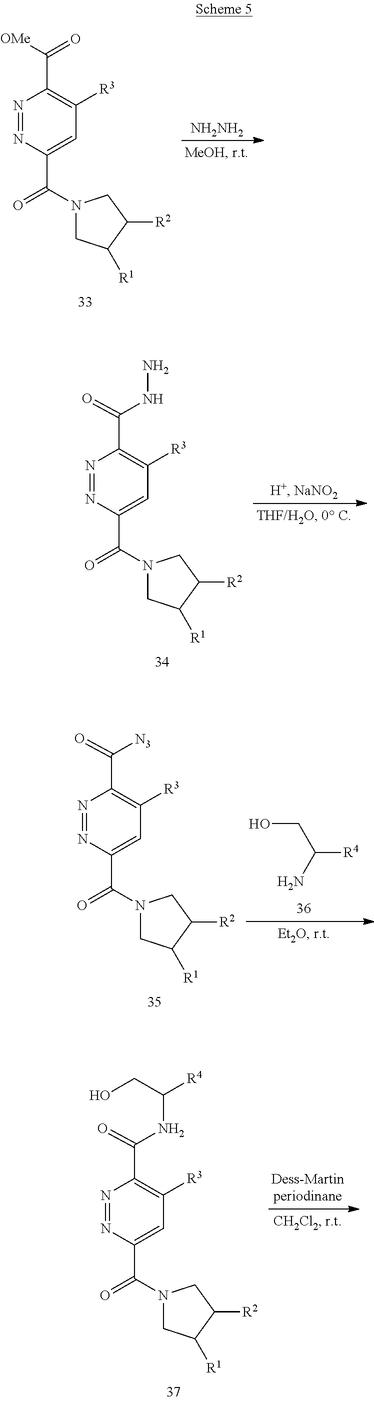

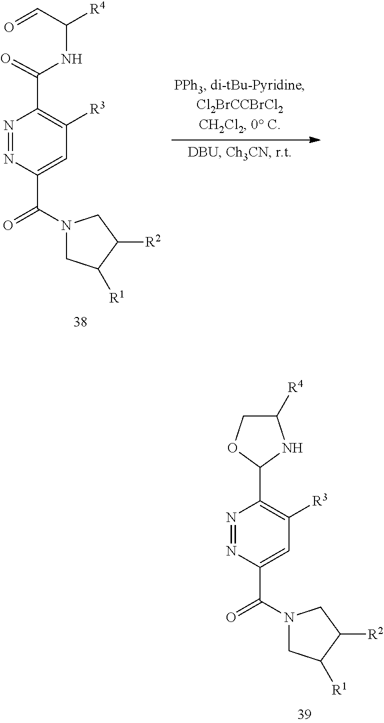

For example, as shown in Scheme 5, the ester function of Cmpd XX can be converted to the corresponding hydrazide in the presence of hydrazine. This hydrazide function can then be diazotized in the presence of NaNO₂ following the Curtius procedure. See: Meienhofer, J. In The peptides, Analysis, Synthesis, Biology; Gross, E., Meienhofer, J., Eds.; Academic Press, Inc., 1979; Vol. 1, pp 197-228) to give the acyl-azide XX which can be trapped by various amino-alcohols XX to give the corresponding amides XX. The primary alcohol of XX can then be smoothly oxidized to the aldehyde XX in the presence of the Dess-Martin periodinane reagent. Compound XX can be converted in a one-pot procedure to the desired oxazole-pyridazine-pyrrolidine scaffold XX by formation of the bromooxazolidine in the presence of PPh₃ and BrCl₂CCCl₂Br followed by elimination of HBr in the presence of DBU.

### IV. Methods of Use

There are provided herein methods of inhibiting or disrupting the interaction between two proteins. In some embodiments, the method includes inhibiting or disrupting the interaction between an alpha helix of a first protein and the alpha helix binding pocket of a second protein. In some embodiments, the method includes the step of contacting the second protein with a compound as described herein. In some embodiments, the protein-protein interaction involves Bak/Bcl-x_{L}, p53/HDM2, calmodulin/smooth muscle myosin light-chain kinase, or the gp41 assembly.

Thus, in one embodiment, a method of treating a disease, condition or disorder mediated by disrupting a protein-protein interaction is provided. The method includes the step of administering a therapeutically effective amount of a compound provided herein to a patient in need thereof to treat the disease, condition or disorder. In some embodiments, the disease is cancer, a viral infection (e.g. HIV infection), or AIDS.

Protein-protein interactions involving an alpha helix of a first protein and a alpha helix pocket of a second protein are well known in the art. Without being limited by any particular theory, the mechanism of binding appears to involve the fitting of the hydrophobic face of a small amphipathic alpha helix of one protein into a well-defined pocket on another protein during their binding to one another. Examples of such interactions include, but are not limited to, protein-protein interactions described herein.

### V. Assays

To develop compounds useful for disrupting protein-protein interactions, candidate inhibitors may be identified in vitro. The activity of the such compounds can be assayed utilizing methods known in the art and/or those methods presented herein. Exemplary methods for detecting disruption of protein-protein interactions include, but are not limited to, circular dichroism, fluorescence energy transfer, fluorescence polarization, ligand displacement, ultracentrifugation, and other methodologies which can assess protein-protein interaction and the disruption thereof by compounds described herein.

An exemplary ligand displacement system relates to galanin, which is a peptide hormone of diverse biological effect found through out the nervous and endocrine systems of a number of species. Galanin binds to at least three different G-protein coupled receptors (GalR1-3) and influences such processes as insulin secretion, gut secretion/motility, memory, sexual behavior, and pain regulation among others. Site-directed mutagenesis studies on a sixteen-amino acid fragment have shown that this peptide binds to galanin receptor type 1 (GalR1) through three amino acid residues (Trp², Asn⁵, Tyr⁹), thought to be in an alpha-helical conformation, as well as through the N-terminal residue. See Branchek et al., Ann. NY Acad. Sci. 1998, 863, 94.

Once compounds are identified that are capable of disrupting protein-protein interactions, the compounds may be further tested for their ability to selectively inhibit specific interactions. Exemplary protein-protein interactions in this respect include, but are not limited to, Bak/Bcl-x_{L}, p53/HDM2, calmodulin/smooth muscle myosin light-chain kinase, and the gp41 assembly. Inhibition by a compound described herein is measured using standard in vitro or in vivo assays such as those well known in the art or as otherwise described herein.

Compounds may be further tested in cell models or animal models for their ability to cause a detectable changes in phenotype related to disruption of a protein-protein interaction.

### VI. Pharmaceutical Compositions

The instant invention also provides for pharmaceutical compositions which may be prepared by mixing one or more compounds of the invention, pharmaceutically acceptable salts thereof, stereoisomers thereof, tautomers thereof, or solvates thereof, with pharmaceutically acceptable carriers, excipients, binders, diluents or the like to treat or ameliorate a variety of disorders mediated by calcitonin and/or amylin receptors. The compositions of the invention may be used to create Formulations and prevent or treat disorders mediated by calcitonin and/or amylin receptors such as bone and metabolic diseases. Such compositions can be in the form of, for example, granules, powders, tablets, capsules, syrup, suppositories, injections, emulsions, elixirs, suspensions or solutions. The instant compositions can be formulated for various routes of administration, for example, by oral administration, by nasal administration, by rectal administration, subcutaneous injection, intravenous injection, intramuscular injections, or intraperitoneal injection. The following dosage forms are given by way of example and should not be construed as limiting the instant invention.

For oral, buccal, and sublingual administration, powders, suspensions, granules, tablets, pills, capsules, gelcaps, and caplets are acceptable as solid dosage forms. These can be prepared, for example, by mixing one or more compounds of the instant invention, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical Formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension Formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension Formulations.

For nasal administration, the pharmaceutical Formulations and medicaments may be a spray or aerosol containing an appropriate solvent(s) and optionally other compounds such as, but not limited to, stabilizers, antimicrobial agents, antioxidants, pH modifiers, surfactants, bioavailability modifiers and combinations of these. A propellant for an aerosol Formulation may include compressed air, nitrogen, carbon dioxide, or a hydrocarbon based low boiling solvent.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical Formulation and/or medicament may also be a powder suitable for reconstitution with an appropriate solution as described above. Examples of these include, but are not limited to, freeze dried, rotary dried or spray dried powders, amorphous powders, granules, precipitates, or particulates. For injection, the Formulations may optionally contain stabilizers, pH modifiers, surfactants, bioavailability modifiers and combinations of these.

For rectal administration, the pharmaceutical Formulations and medicaments may be in the form of a suppository, an ointment, an enema, a tablet or a cream for release of compound in the intestines, sigmoid flexure and/or rectum. Rectal suppositories are prepared by mixing one or more compounds of the instant invention, or pharmaceutically acceptable salts or tautomers of the compound, with acceptable vehicles, for example, cocoa butter or polyethylene glycol, which is present in a solid phase at normal storing temperatures, and present in a liquid phase at those temperatures suitable to release a drug inside the body, such as in the rectum. Oils may also be employed in the preparation of Formulations of the soft gelatin type and suppositories. Water, saline, aqueous dextrose and related sugar solutions, and glycerols may be employed in the preparation of suspension Formulations which may also contain suspending agents such as pectins, carbomers, methyl cellulose, hydroxypropyl cellulose or carboxymethyl cellulose, as well as buffers and preservatives.

Besides those representative dosage forms described above, pharmaceutically acceptable excipients and carriers are generally known to those skilled in the art and are thus included in the instant invention. Such excipients and carriers are described, for example, in Remingtons Pharmaceutical Sciences, Mack Pub. Co., New Jersey (1991), which is incorporated herein by reference.

The formulations of the invention may be designed to be short-acting, fast-releasing, long-acting, and sustained-releasing as described below. Thus, the pharmaceutical Formulations may also be Formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical Formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant invention.

A therapeutically effective amount of a compound of the present invention may vary depending upon the route of administration and dosage form. The typical compound or compounds of the instant invention is a formulation that exhibits a high therapeutic index. The therapeutic index is the dose ratio between toxic and therapeutic effects which can be expressed as the ratio between LD₅₀ and ED₅₀. The LD₅₀ is the dose lethal to 50% of the population and the ED₅₀ is the dose therapeutically effective in 50% of the population. The LD₅₀ and ED₅₀ are determined by standard pharmaceutical procedures in animal cell cultures or experimental animals.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

The present invention, thus generally described, will be understood more readily by reference to the following examples, which are provided by way of illustration and are not intended to be limiting of the present invention.

### VII. Examples

General Methods. Commercially available reagent-grade solvents were employed without purification. ¹H and ¹³C NMR spectra were recorded on 300 or 600 MHz spectrometers. Chemical shifts are expressed in ppm (δ), using tetramethylsilane (TMS) as internal standard for ¹H and ¹³C nuclei (δ_{H} and δ_{C}=0.00).

Abbreviations. The following abbreviations are used herein: AcOH: Acetic acid; BuOH: Butanol; cHex: Cyclohexane; DAST: N,N-Diethylaminosulfur trifluoride; DCM: dichloromethane; DIEA: N,N-Diisopropylethylamine; DMF: N,N-Dimethylformamide; DMSO: Dimethylsulfoxide; EDCI: 1-ethyl-3-(3′-dimethylaminopropyl)carbodiimide hydrochloride; EtOAc: ethyl acetate; Et₃N, TEA: Triethylamine; HATU: 2-(7-Aza-1H-benzotriazole-1-yl)-1,1,3,3-tetramethyluronium hexafluorophosphate; Hex: Hexanes; HOBt: 1-Hydroxybenzotriazole; MeOH: Methanol; m.p.: Melting point; NMR: Nuclear magnetic resonance; r.t.: Room temperature; TFA: Trifluoroacetic acid; THF: Tetrahydrofuran; TMS: Trimethylsilyl.

### Example 1

### 4-Isobutyl-6-(pyrrolidine-1-carbonyl)-pyridazine-3-carboxylic acid methyl ester

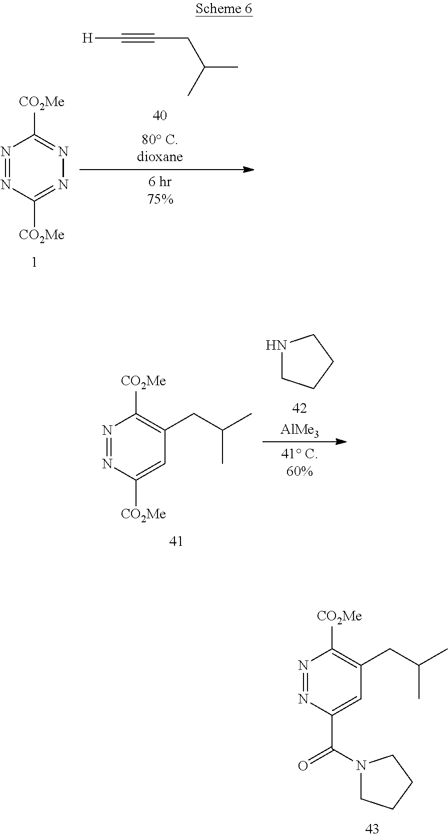

4-Isobutyl-pyridazine-3,6-dicarboxylic acid dimethyl ester. As shown in Scheme 6, tetrazine 1 (obtained for example as described in Boger. et al. Org. Synth. 1992, Id.; Spencer et al. J. Chem. Phys. 1961, 35, 1939; Sauer J. et al. Chem. Ber. 1965, 98, 1435) (500 mg, 2.52 mmol) can be dissolved in 12.5 mL anhydrous 1,4-dioxane. To this bright red solution can be added 355 μL 4-methyl pentyne (234 mg, 2.85 mmol), after which the reaction vessel can be sealed and heated to 80° C. for 6 hours. The volatiles can be removed under reduced pressure, and the crude product can be purified via silica gel chromatography (9.5:0.5 CH₂Cl₂-EtOAc) to afford Cmpd 41.

4-Isobutyl-6-(pyrrolidine-1-carbonyl)-pyridazine-3-carboxylic acid methyl ester. An oven dried, nitrogen cooled two-necked 25 mL round bottom flask can be charged with a solution of pyrrolidine (42, about 0.8 mmol) in 4.0 mL anhydrous DCM. Trimethyl aluminum can be added slowly (2.0M/toluene, 436 μL, 0.872 mmol). The resulting mixture can be stirred for twenty minutes at room temperature. To this solution can be added 200 mg of the diester Cmpd 41 (0.793 mmol) dissolved in 2.0 mL anhydrous DCM. The flask can be sealed and heated to 41° C. for 15 hours. The reaction can be allowed to cool to room temperature and quenched with slow addition of 1M HCl. The resultant can be extracted multiple times with DCM, the organic fractions can be collected, washed with brine, dried over magnesium sulfate and evaporated to give a crude mixture. The crude mixture can be applied to a column of silica gel and eluted with a 2:1 hexane-ethyl acetate solution to afford Cmpd 43.

### Example 2

### 4-Isobutyl-6-(pyrrolidine-1-carbonyl)-pyridazine-3-carboxylic acid ((S)-1-hydroxymethyl-2-methyl-propyl)-amide

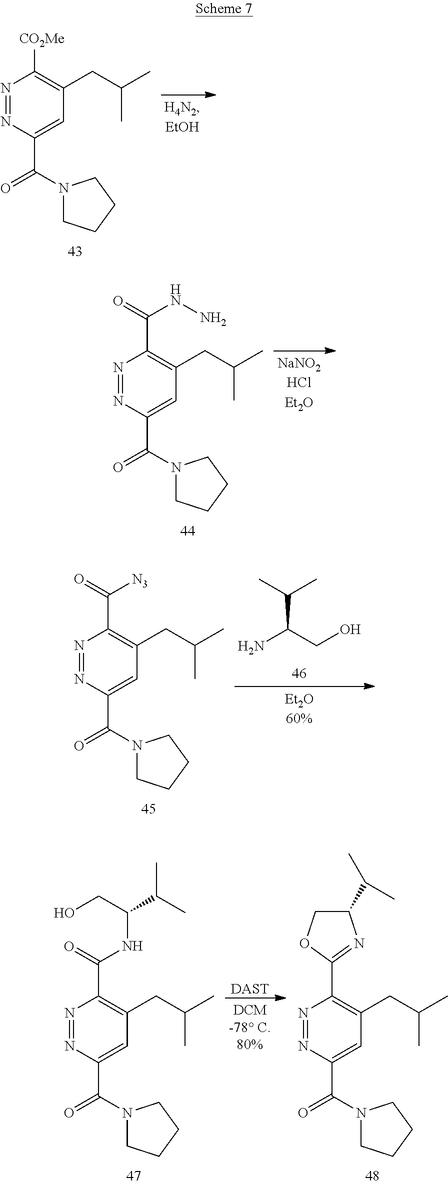

4-Isobutyl-6-(pyrrolidine-1-carbonyl)-pyridazine-3-carboxylic acid hydrazide. As shown in Scheme 7, the mono-methyl ester Cmpd 43 (about 0.033 mmol) can be dissolved in 3.5 mL anhydrous ethanol (dried over 4 Å molecular sieves). Hydrazine hydrate can be added (55 μL, 1.65 mmol, 53 mg), and the reaction can be stirred for 5.5 hours at room temperature under an atmosphere of nitrogen. The volatiles can be evaporated under reduced pressure, and the product oil can be triturated three times with about 3 mL diethyl ether. The resultant can be dried under high vacuum to Cmpd 44.

4-Isobutyl-6-(pyrrolidine-1-carbonyl)-pyridazine-3-carboxylic acid ((S)-1-hydroxymethyl-2-methyl-propyl)-amide. Sodium nitrite (12 mg, 0.164 mmol) can be dissolved in 7.5 mL H₂O. The solution can be cooled to 0° C., and 150 μL 1.2M HCl (0.180 mmol) can be added slowly. The mixture can be allowed to stir for 10 minutes, at which time a solution of 50 mg hydrazide Cmpd 44 (0.164 mmol) in 9 mL ethyl ether can be added dropwise. The mixture can be stirred vigorously for another 10 minutes at 0° C. The more non-polar acyl-azide can be visualized by TLC, 2:2:0.4 Hex-EtOAc-MeOH. The aqueous layer can be removed, and a pre-cooled solution of L-valinol Cmpd 46 (18.5 mg, 0.180 mmol) in 2 mL ethyl ether can be added slowly. The reaction can be allowed to warm to room temperature. The reaction mixture can be diluted with ethyl ether, and the organic solution can be washed with 1M HCl (3×5 mL), saturated NaHCO₃ (3×5 mL), brine (1×5 mL), dried over MgSO₄ and evaporated under reduced pressure afford a crude product. The crude product can be purified using silica gel chromatography (4:4:0.4 Hex-EtOAc-MeOH, R_{f}: 0.21) to afford Cmpd 47.

(S)-(5-Isobutyl-6-(4-isopropyl-4,5-dihydrooxazole-2-yl)pyridazin-3-yl) (pyrrolidin-1-yl)methanone (9). β-Hydroxy amide Cmpd 47 (about 0.013 mmol) can be dissolved in 250 μL anhydrous DCM and cooled to −78° C. (CO₂/acetone). DAST (2.0 μL, 0.014 mmol, 2.3 mg) can be added, and the reaction mixture can be stirred at −78° C. for 1 hour. Potassium carbonate (3 mg, 0.020 mmol) can be added in one portion, and the solution can be allowed to warm to room temperature. A solution of saturated sodium bicarbonate can be added (0.5 mL), and the resultant compound can be extracted with DCM (3×1 mL). The organic fractions can be collected, washed with brine (1×2 mL), dried over Na₂SO₄, and evaporated to dryness. The crude product can be purified using preparatory silica gel chromatography (10 cm×20 cm×250 μm; 2:2:0.4 EtOAc-Hex-MeOH) to afford oxazoline Cmpd 48.

### Example 3

### Synthesis of dimethyl 4-((1H-indol-3-yl)methyl)pyridazine-3,6-dicarboxylate

As a first approach to compound 2a, the diester 1 (Boger, D. L.; et al. Org. Synth. 1992, Id.; Naud, S. Synlett 2004, 2836-2837) was reacted with the commercially available 2-methoxy-3,4-dihydro-2H-pyrane to give pyridazine 4 in 71% yield. Subsequent treatment under standard Fischer indole synthesis (Hutchins, S. M.; Chapman, K. T. Tetrahedron Lett. 1996, 37, 4869-4872) conditions led predominantly to the decomposition of the starting material and formation of the desired unprotected compound 2a in 13% yield. See Scheme 8.

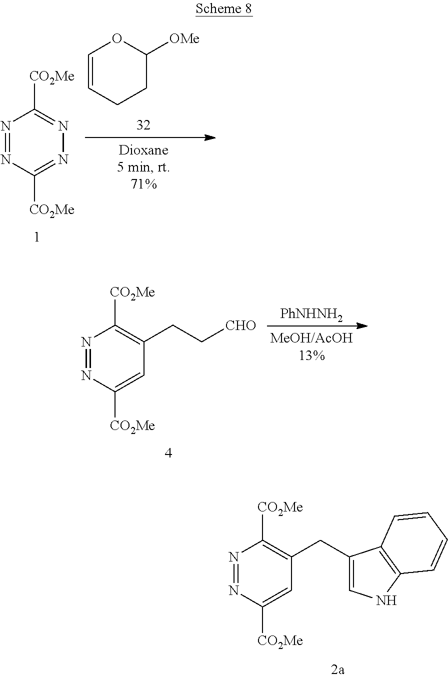

### Example 4

### Synthesis of dimethyl 4-((1-(tert-butoxycarbonyl)-1H-indol-3-yl)methyl)pyridazine-3,6-dicarboxylate (2a)

The N-Boc protected derivative 5 of the commercial available methyl-2-(1H-indol-3-yl)acetate was selected as the precursor of the electron rich dienophile 6. See Scheme 9. Treatment of compound 5 with the Tebbe reagent in tetrahydrofuran at low temperature afforded the desired enol ether 6, and reaction with tetrazine 1 at room temperature to afford pyridazine 2a in 43% overall yield from compound 5.

Representative procedure: To a solution of the corresponding ester (1.04 mmol) in THF (12 mL) cooled at −40° C. is added Tebbe reagent (2.7 mL, 1.3 eq., 0.5 M in toluene). After 30 min the temperature is raised to ambient over a period of 2 hours. The mixture is then cooled to −10° C. and the reaction is quenched by the dropwise addition of NaOH (700 μL, 2 M solution). Reaction mixture is then allowed to warm to room temperature. The solution is then diluted with excess of ether and filtered through a pad of Celite®. Solvent is removed under reduced pressure and the crude residue is directly diluted in dioxane (10 mL) and added to a solution of tetrazine 1 (203 mg, 1.0 mmol) in dioxane (10 mL). After 18 hours at room temperature, volatiles are removed and the crude residue is purified on silica gel (hexane/AcOEt mixtures) to afford the desired compound.

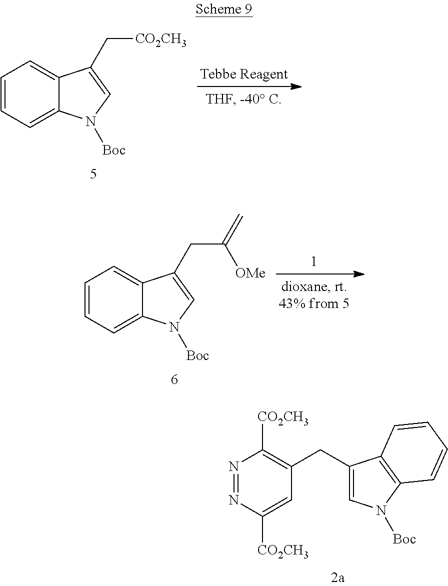

### Example 5

### Synthesis of Intermediates Via Reaction with Tebbe Reagent

The scope of the method described above was expanded with several commercially available esters that were subjected to the synthetic sequence shown in Scheme 9. See Table 1 following. The Tebbe reaction is compatible with the presence of a variety of functional groups such as carbamates (2f-g), ethers (2d), thioethers (2h-i) or sterically challenged esters (2e), allowing the preparation of pyridazines containing substituents that are conveniently protected side chains of functional amino acids. Various compounds containing either aromatic or aliphatic substituents in position 4 of the pyridazine core were prepared. The best yields were obtained with esters with aromatic rings in their structures (entries 1-4). For example, the synthesis of compound 2e, which features a tert-butyl ester of a glutamic acid residue, began with the commercially available tert-butyl methyl glutarate (entry 5). The Tebbe reagent showed exclusive regioselectivity, giving the enol ether of the less hindered methyl ester.

TABLE 1
Selected starting materials, intermediates and yields.

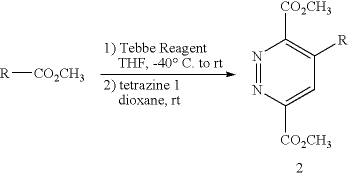

| No. | Ester | Product | | Yield (%) |
|---|---|---|---|---|
| 1 | 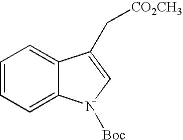 | 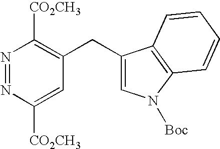 | 2a | 43 |
| 2 | 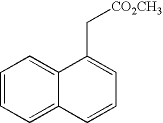 | 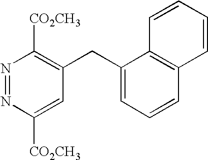 | 2b | 65 |
| 3 | 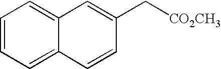 | 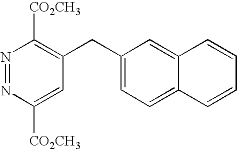 | 2c | 54 |
| 4 | 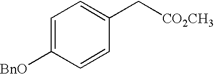 | 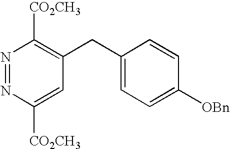 | 2d | 58 |
| 5 | 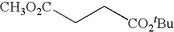 | 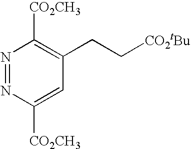 | 2e | 41 |
| 6 | 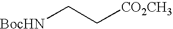 | 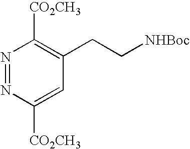 | 2f | 39 |
| 7 | 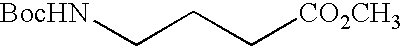 | 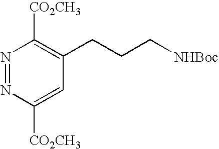 | 2g | 31 Cmpd 1 limiting |
| 8 | 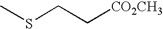 | 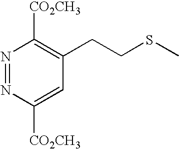 | 2h | 41 |
| 9 | 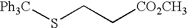 | 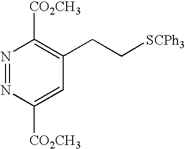 | 2i | 35 Cmpd 1 limiting |

### Example 6

### Representative Synthesis of Analogs of Cmpd 15

Compounds having the general structural features of Cmpd 15 can be synthesized by the route provided in Scheme 10 following.

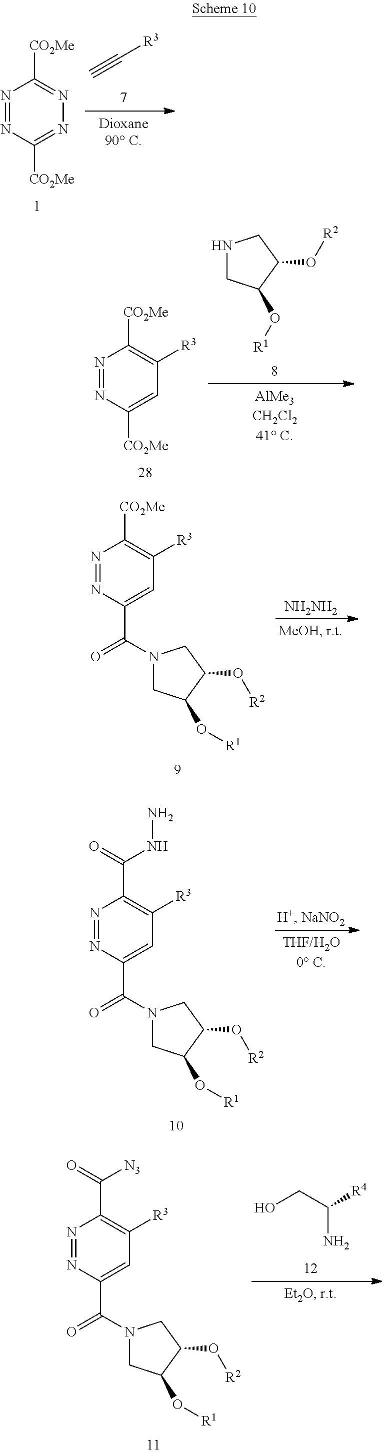

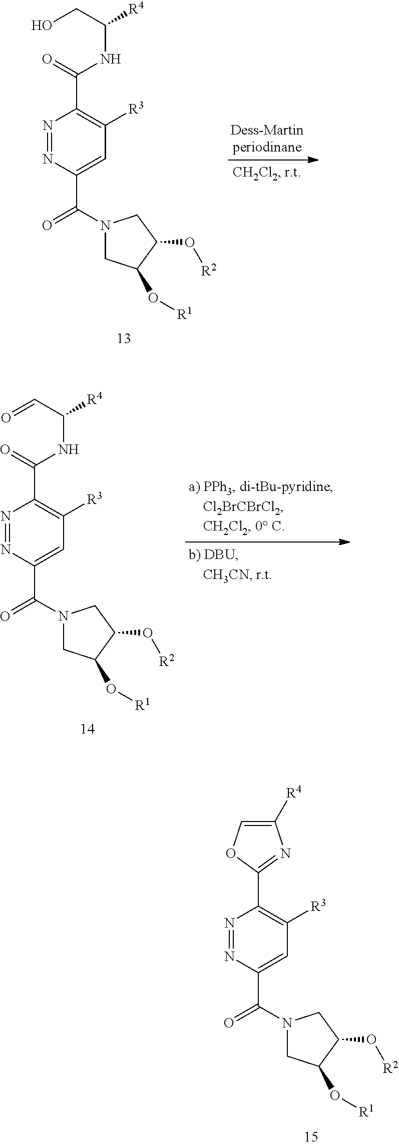

As shown in Scheme 10, Tetrazine 1 is prepared from ethyl diazoacetate according to the procedure described by Boger, et al., (D. Boger, et al., Org. Synth., 1992, Id.). Heated in the presence of a dienophile, tetrazine 1 can undergo an inverse demand Diels-Alder reaction to give pyridazine 2. Dienophiles are typically substituted alkynes but can also be enol-ether or enamines (D. Boger, Tetrahedron, 1983, 39, 2869). Substituted (S,S)-pyrrolidine 8 can then be coupled regioselectively in the presence of AlMe₃ according to the procedure described by Weinreb (A. Basha, et al., Tetrahedron Lett. 1977, 48, 4171). The remaining ester function in compound 9 can then be converted to the corresponding hydrazide 10 in the presence of hydrazine. This hydrazide function can then be diazotized in the presence of NaNO₂ following the Curtius procedure. See: Meienhofer, J. In The peptides, Analysis, Synthesis, Biology; Gross, E., Meienhofer, J., Eds.; Academic Press, Inc., 1979; Vol. 1, pp 197-228) to give the acyl-azide 11 which can be trapped by various amino-alcohols 12 to give the corresponding amides 13. The primary alcohol of 13 can then be smoothly oxidized to the aldehyde 14 in the presence of the Dess-Martin periodinane reagent. Compound 14 can be converted in a one-pot procedure to the desired oxazole-pyridazine-pyrrolidine scaffold 15 by formation of the bromooxazolidine in the presence of PPh₃ and BrCl₂CCCl₂Br followed by elimination of HBr in the presence of DBU.

### Example 7

### Representative Synthesis of Substituted Pyrrolidines

Pyrrolidine 8 is obtained in three steps from pyrrolidine 16 using two successive alkylations with alkyl iodides and a final deprotection of the secondary nitrogen with Pd/C under a H₂ atmosphere. See Scheme 11.

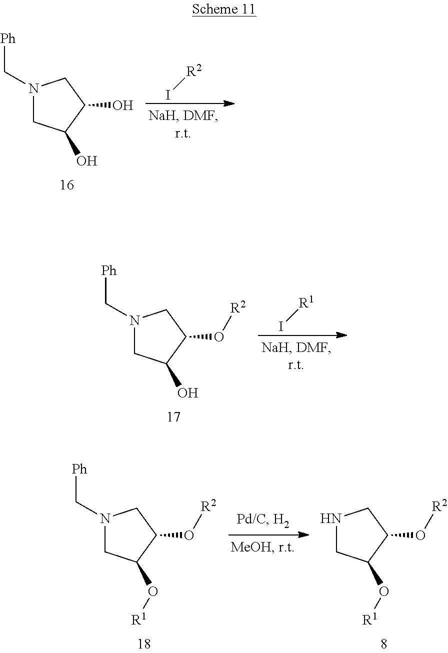

Pyrrolidine 16 is commercially available as well as the other corresponding diastereoisomers that can also be used in this synthesis. Alternative coupling procedures such as activation of the methyl ester function with mild lewis acid (typically MgCl₂, see: González-Gómez, J. C.; Santana, L.; Uriarte, E. Tetrahedron 2003, 59, 8171) or hydrolysis of the methyl ester function in the presence of LiOH followed by standard peptide coupling conditions are also possible for this step.

### Example 8

### Additional Compounds

Exemplary compounds having a functionalized pyridazine as exemplified by the formula of Cmpd 15 are provided in Table 2 following.

TABLE 2
Exemplary compounds

| Cmpd | No. |
|---|---|
| 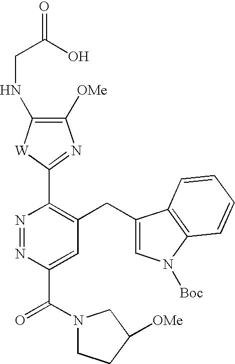 | 49 |
| 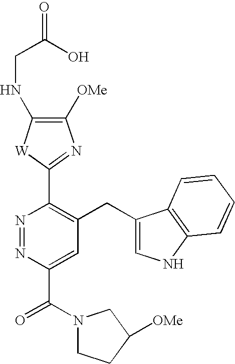 | 49′ |
| 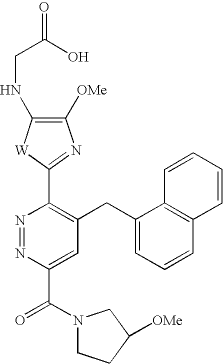 | 50 |
| 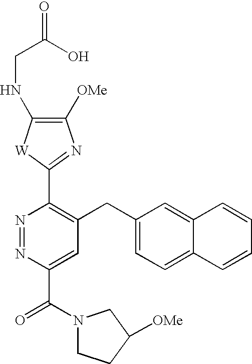 | 51 |
| 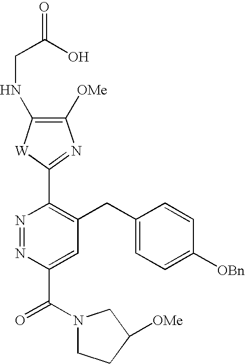 | 52 |
| 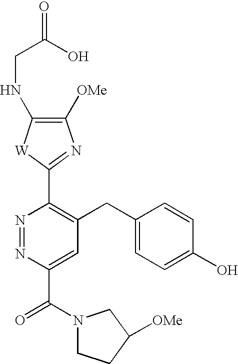 | 52′ |
| 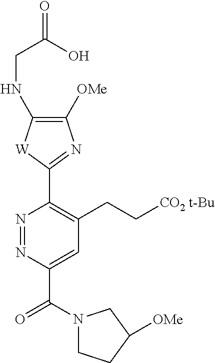 | 53 |
| 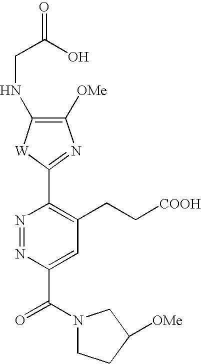 | 53′ |
| 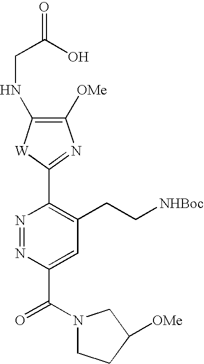 | 54 |
| 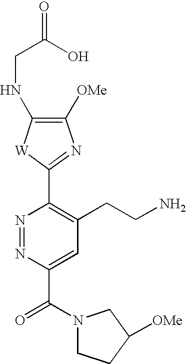 | 54′ |
| 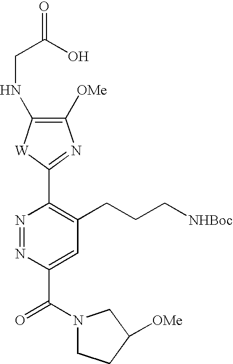 | 55 |
| 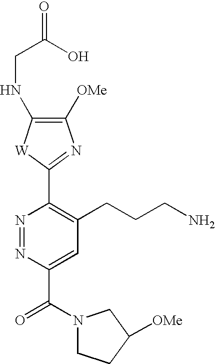 | 55′ |
| 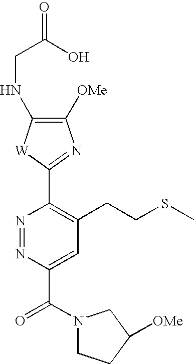 | 56 |
| 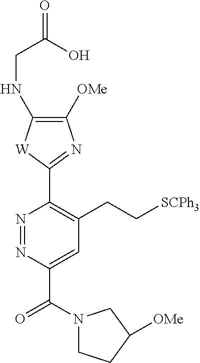 | 57 |
| 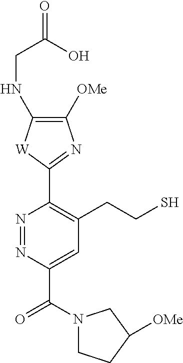 | 57′ |

### Example 9

### Biological Assays

Galanin Assay Compounds of the present invention can be tested for binding affinity to GalR1 using the protocol described by Land et al., Methods Neurosci. 1991, 5, 225. Many compounds of the invention will demonstrate binding to GalR1. Compounds of the present invention can be tested for ability to displace [¹²⁵I]-Galanin from mice hippocampus membranes (which contain all of the Galanin receptors) also according to the procedure of Land. Many such compounds will demonstrate the ability to displace [¹²⁵I]-Galanin from mice hippocampus membranes.

Bcl-x_{L}-Bak fluorescence polarization assay. The binding affinity of the molecules for Bcl-x_{L} can be assessed by a fluorescence polarization assay using fluorescein-labeled 16-mer Bak-peptide [A. M. Petros et al. Protein Science. 2000, 9, 2528]. Displacement of this probe through competitive binding of the compounds into the hydrophobic cleft of Bcl-x_{L} would lead to a decrease in its fluorescence polarization which in turn can be related to the known affinity of the 16-mer Bak/Bcl-x_{L} complex.

### Example 10

### Formulations

Solution for Parenteral Administration: A solution is prepared from the following ingredients:

| | |
|---|---|
| Active compound | 5 g |
| Sodium chloride for injection | 6 g |
| Sodium hydroxide | for pH adjustment at pH 5-7 |
| Water for injection. | Up to 1000 mL |

The active constituent and the sodium chloride are dissolved in the water. The pH is adjusted with 2M NaOH to pH 3-9 and the solution is filled into sterile ampoules.

Tablets for Oral Administration: 1000 tablets are prepared from the following ingredients:

| Component | Amount |
|---|---|
| Active compound | 100 g |
| Lactose | 200 g |
| Polyvinyl pyrrolidone | 30 g |
| Microcrystalline cellulose | 30 g |
| Magnesium stearate | 6 g |

The active constituent and lactose are mixed with an aqueous solution of polyvinyl pyrrolidone. The mixture is dried and milled to form granules. The microcrystalline cellulose and then the magnesium stearate are then admixed. The mixture is then compressed in a tablet machine giving 1000 tablets, each containing 100 mg of active constituent.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, any group having 1-3 atoms refers to groups having 1, 2, or 3 atoms. Similarly, a group having 1-5 atoms refers to groups having 1, 2, 3, 4, or 5 atoms, and so forth.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

While preferred embodiments have been illustrated and described, it should be understood that changes and modifications can be made therein in accordance with ordinary skill in the art without departing from the invention in its broader aspects as defined in the following claims.

What is claimed is:

1. A compound having the structure of Formula (I) 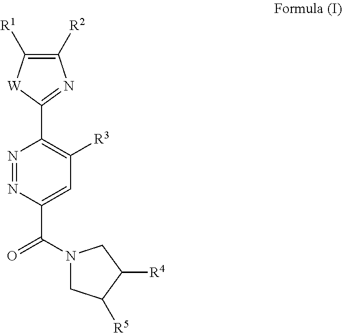
wherein:
W is —O— or —S—;
R¹ and R² are independently hydrogen, substituted or unsubstituted alkyl, substituted or unsubstituted heteroalkyl, or a side chain of a naturally occurring amino acid, homolog thereof, or chemically protected analog thereof, optionally linked through an —O— ether linkage, provided that at least one of R¹ and R² is a side chain of a naturally occurring amino acid, homolog thereof, or chemically protected analog thereof, optionally linked through an —O— ether linkage;
R³ is hydrogen, substituted or unsubstituted alkyl, or a side chain of a naturally occurring amino acid, homolog thereof, or chemically protected analog thereof; and
R⁴ and R⁵ are independently hydrogen, substituted or unsubstituted alkyl, substituted or unsubstituted heteroalkyl, or a side chain of a naturally occurring amino acid, homolog thereof, or chemically protected analog thereof, optionally linked through an —O— ether linkage, provided that at least one of R⁴ and R⁵ is a side chain of a naturally occurring amino acid, homolog thereof, or chemically protected analog thereof, optionally linked through an —O— ether linkage.

2. The compound according to claim 1, provided that, at most, only one of R¹ and R², R³, and R⁴ and R⁵ is hydrogen.

3. The compound according to claim 1,
wherein:
R¹ and R² are independently hydrogen, substituted or unsubstituted alkyl, substituted or unsubstituted heteroalkyl, or a side chain of a naturally occurring amino acid, homolog thereof, or chemically protected analog thereof, provided that at least one of R¹ and R² is a side chain of a naturally occurring amino acid, homolog thereof, or chemically protected analog thereof;
R⁴ and R⁵ are independently hydrogen, substituted or unsubstituted alkyl, substituted or unsubstituted heteroalkyl, or a side chain of a naturally occurring amino acid, homolog thereof, or chemically protected analog thereof, provided that at least one of R⁴ and R⁵ is a side chain of a naturally occurring amino acid, homolog thereof, or chemically protected analog thereof.

4. The compound according to claim 1,
wherein:
R¹ and R² are independently a side chain of a naturally occurring amino acid, homolog thereof, or chemically protected analog thereof,
R³ is a side chain of a naturally occurring amino acid, homolog thereof, or chemically protected analog thereof; and
R⁴ and R⁵ are independently a side chain of a naturally occurring amino acid, homolog thereof, or chemically protected analog thereof.

5. The compound according to claim 1, wherein R³ is selected from the group consisting of -(unsubstituted C₁-C₉ alkyl), —CH₂-(unsubstituted C₃-C₈ cycloalkyl), —CH₂-(unsubstituted C₆-C₁₀ aryl), —(CH₂)₂—SCPh₃, —(CH₂)₂—SMe, —(CH₂)₃—NBoc, —(CH₂)₂—NBoc, —(CH₂)₂—CO₂-^{t}Bu,

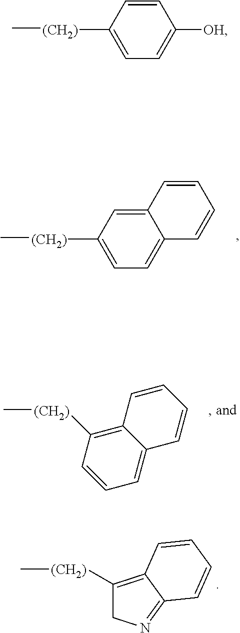

6. The compound according to claim 1, wherein either of R⁴ and R⁵ are selected from the group consisting of —O-(unsubstituted C₁-C₆ alkyl), —O-(unsubstituted C₁-C₆ alkyl)-CO₂H, —O—C(O)-(unsubstituted C₁-C₆ alkyl), and —O—C(O)-(unsubstituted C₁-C₆ alkyl)-CO₂H.

7. The compound according to claim 1, wherein either of R⁴ and R⁵ are selected from the group consisting of -(unsubstituted C₁-C₆ alkyl), -(unsubstituted C₁-C₆ alkyl)-CO₂H, —C(O)-(unsubstituted C₁-C₆ alkyl), and —C(O)-(unsubstituted C₁-C₆ alkyl)-CO₂H.

8. The compound according to claim 1 wherein the side chain of the naturally occurring amino acid with respect to R¹, R², R³, R⁴, and R⁵ is a radical selected from the group of radicals consisting of —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —H₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, —O(C₁-C₆ unsubstituted alkyl), —OC(O)—(C₁-C₆ unsubstituted alkyl), and homologs thereof.

9. The compound according to claim 1 having the following structure:

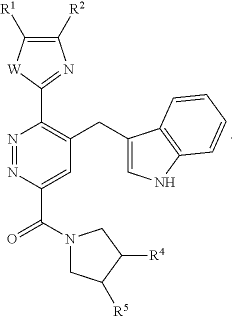

10. The compound according to claim 1 having the following structure:

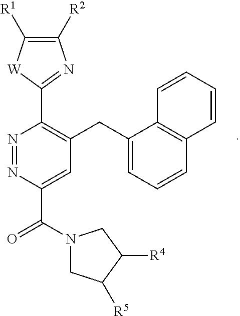

11. The compound according to claim 1 having the following structure:

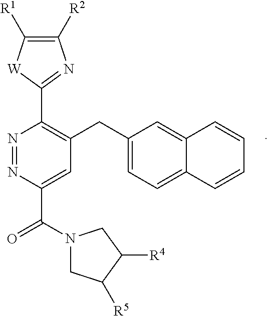

12. The compound according to claim 1 having the following structure:

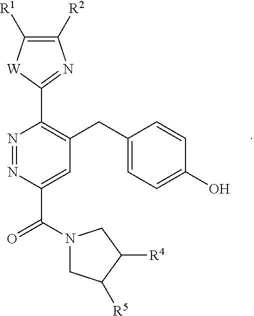

13. The compound according to claim 1 having the following structure:

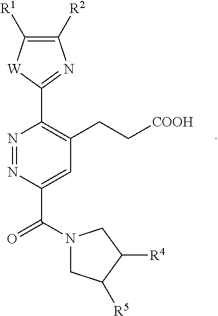

14. The compound according to claim 1 having the following structure:

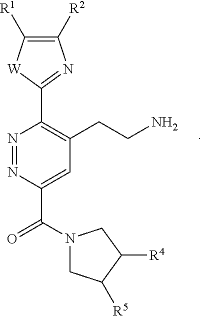

15. The compound according to claim 1 having the following structure:

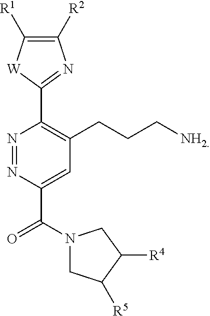

16. The compound according to claim 1 having the following structure:

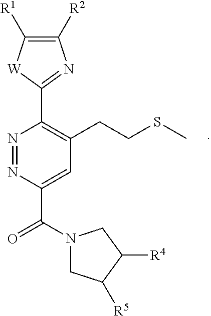

17. A compound according to claim 1 represented by the following structure:

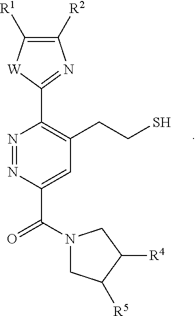

18. A process for synthesizing any of the compounds of claims 1-17 and intermediates thereof.

19. A method for disrupting a protein-protein interaction selected from the group consisting of Bak/Bcl-x_{L}, p53/HDM2, calmodulin/smooth muscle myosin light-chain kinase, and gp41 assembly, said method comprising the step of contacting a protein involved in said protein-protein interaction with the compound of claim 1.

20. A method for treating a disease, conditions and/or disorder mediated by a protein-protein interaction, wherein said protein-protein interaction is selected from the group consisting of Bak/Bcl-x_{L}, p53/HDM2, calmodulin/smooth muscle myosin light-chain kinase, and gp41 assembly, said method comprising the step of administering to a patient in need thereof a therapeutically effective amount of the compound of claim 1.
